(19) United States
(12) Patent Application Publication
(10) Pub. No.: US 2025/0087306 A1
(43) Pub. Date: Mar. 13, 2025

(54) PROCESSES FOR MODULATING PLANT GENE EXPRESSION

(71) Applicant: PHYTOFORM LABS LTD., Harpenden Hertfordshire (GB)

(72) Inventors: William Pelton, Harpenden Hertfordshire (GB); Sania Jevtic, Harpenden Hertfordshire (GB); Timo Flesch, Harpenden Hertfordshire (GB); Andrew Brockman, Harpenden Hertfordshire (GB); Nicolas Kral, Harpenden Hertfordshire (GB)

(21) Appl. No.: 18/955,131

(22) Filed: Nov. 21, 2024

Related U.S. Application Data
(63) Continuation of application No. PCT/EP2023/063847, filed on May 23, 2023.

(30) Foreign Application Priority Data
May 23, 2022 (GB) 2207533.7

(51) Int. Cl. G16B 35/10; C12N 15/10; C12N 15/82; C40B 40/06; G06N 3/0464

(52) U.S. Cl. CPC G16B 35/10; C12N 15/1086; C12N 15/8216; C40B 40/06; G06N 3/0464

(57) ABSTRACT

Methods are provided for performing an in silico analysis of a genome for a plant subject, that includes identification of a plurality of sequences that collectively define a promoterome of the plant subject, wherein each sequence within the promoterome is comprised of an expression control region that extends from the start codon of an open reading frame to around 100 kilobases 5′ and/or 3′ to the start codon. A transcriptome in the form of mRNA expression data for the plant subject is obtained and an analysis is undertaken using a sequence-based modelling algorithm in order to provide a prediction value of the level of expression of each protein coding gene comprised within the transcriptome and linking the value to a sequence for a corresponding expression control region comprised within the promoterome. The methods may be used to generating novel designs for expression control sequences that are most likely to provide a desired expression profile for an operably linked coding sequence. Also provided are nucleic acid libraries, plant cells, plant tissues and plants comprising novel expression control sequences.

### CROSS-REFERENCE TO RELATED APPLICATIONS

This application is a continuation of PCT/EP2023/063847, filed May 23, 2023; which claims the benefit of Great Britain Application No. 2207533.7, filed May 23, 2022. The contents of the above-identified applications are incorporated herein by reference in their entireties.

### SEQUENCE LISTING

This application contains an ST.26 compliant Sequence Listing, which was submitted in xml format via Patent Center and is hereby incorporated by reference in its entirety. The .xml copy, created on May 22, 2023 is named “P68925WO_SEQ_LIST.xml” and is 16000 bytes in size.

### FIELD OF THE INVENTION

The invention relates to the field of in silico design with optional in vivo validation of novel plant genetic regulatory sequences.

### BACKGROUND OF THE INVENTION

Plants show an amazing capacity for phenotypic plasticity that arises from changes in their genomes brought about by spontaneous mutation, DNA transposition, polyploidization, or crosses with distant relatives. Farmers have been developing new plant varieties since the beginning of agriculture to favour traits such as improved crop yields and disease resistance by exploiting this plasticity. By iteratively selecting for desired traits, such as increased seed or fruit size, species have been phenotypically transformed over time to provide the wide range of varieties and cultivars used in much of modern agriculture. Beginning in the latter part of the last century, knowledge of plant genomes increasingly guided efforts to create new crop varieties. Modern breeding, however, still relies heavily on genetic diversity that occurs spontaneously in nature.

In the last few decades, recombinant genetic manipulation methods have come to the fore that allow for the creation of targeted genetic variations, thereby no longer relying upon natural variation that occurs fortuitously. One such method is transgenesis, which involves the addition of new genes to plant genomes that confer desirable traits such as herbicide resistance. With few exceptions, however, transgenesis has been deployed in only a very few high-value crops and has been considered controversial in some countries. Genetic variation can also be created using mutagenic chemicals such as ethyl methanesulfonate or high energy radiation to alter the genome of the given crop. A drawback of mutation breeding is that it provides very little control and requires extensive mutant populations to be generated and screened in the hope of identifying the very few genetic variations of value. New more targeted mutagenic methods such as/CAS-genome editing offer greater precision, but do not provide a clear understanding of what the valuable outcomes of mutagenesis are.

Prior art approaches have sought to undertake so-called rational design of promoter elements within plant cells using an intuitive researcher-led approach (see Yang et al. (2021) Plant Biotech. J. 19: 1364-1369). This methodology is very much trial and error and is heavily biased towards a narrow range of existing solutions that are known to the researchers from the literature and from within the existing natural variation. These approaches also typically rely on alignment methods for de novo motif discovery, such as motif based sequence analysis tools like meme-suite (mem-suite.org) thereby rationally building a narrow range of solutions. Hence, these techniques significantly reduce the degrees of freedom that may exist for identifying more subtle or non-intuitive positive or negative regulatory changes.

It is desirable, therefore, to provide new methods for developing ways of improving specific traits in plants. In particular, it would be desirable to improve such traits through development of novel plant genetic regulatory sequences, beyond that of natural or mutagenic variation, that control plant gene expression so as to provide such desirable traits.

These and other uses, features and advantages of the invention should be apparent to those skilled in the art from the teachings provided herein.

### SUMMARY OF THE INVENTION

The present inventors have provided new methods that leverage a novel informatics driven approach to driving evolution of novel expression control sequences in plants. These novel sequences may be further validated using in vivo screening methods.

Accordingly, a first aspect of the invention provides a method for producing a nucleic acid library that is comprised of a plurality of expression control sequences that are configured to modulate the expression of coding sequence operably linked thereto in a plant cell, the method comprising:
performing an in silico analysis of a genome for a plant subject, wherein the in silico analysis includes identification of a plurality of sequences that collectively define a promoterome of the plant subject, wherein each sequence within the promoterome is comprised of an expression control region that extends from the start codon of an open reading frame to around 100 kilobases 5′ and/or 3′ to the start codon;
obtaining a transcriptome in the form of mRNA expression data for the plant subject;
undertaking an analysis using a sequence-based modelling algorithm in order to provide a prediction value of the level of expression of each protein coding gene comprised within the transcriptome and linking the value to a sequence for a corresponding expression control region comprised within the promoterome;
generating a plurality of non-wild type sequence designs for expression control sequences that are most likely to provide a desired expression profile for an operably linked coding sequence, wherein plurality of non-wild type sequence designs are informed by the prediction value;
synthesising a plurality of non-wild type expression control sequences that correspond to the plurality of non-wild type sequence designs; and
generating a nucleic acid sequence library that comprises the plurality of non-wild type expression control sequences.

A second aspect of the invention provides a nucleic acid library that comprises a plurality of non-wild type expression control sequences that are configured to modulate the expression of coding sequence operably linked thereto within a plant cell. Suitably, the plurality of non-wild type expression control sequences are generated by the method as defined herein.

Plant cells, protoplasts, plant tissues, calli, plantlets, seeds, and whole plants and other biological materials comprising the nucleic acid library or component sequences thereof are also provided in various aspects and embodiments of the invention.

A third aspect of the invention provides a method, that may be implemented fully or partially on a computer, for performing an analysis of a genome for a plant subject, wherein the analysis comprises:
identification of a plurality of sequences that collectively define a promoterome data set of the plant subject, wherein each sequence within the promoterome data set is comprised of sequence data that corresponds to an expression control region that extends from the start codon of an open reading frame within the genome to around 100 kilobases 5′ and/or 3′ to the start codon;
obtaining a transcriptome data set in the form of mRNA expression data for the plant subject;
undertaking an analysis using a sequence-based machine learning modelling algorithm that is trained with all or a part of the promoterome data set and all or a part of the transcriptome data set in order to provide a prediction value for a level of expression of a gene placed under operative control of any given expression control sequence comprised within the promoterome; and querying the sequence-based machine learning modelling algorithm with one or more query sequence designs, wherein the sequence-based machine learning modelling algorithm applies the prediction value to the one or more query sequence designs in order to provide a prediction of the expression of a gene within the plant subject if the one or more query sequence designs were to be introduced into an expression control region of the gene.

A fourth aspect of the invention provides a for method for generating a modified plant cell comprising performing an analysis of a genome for the plant cell as described herein, identifying at least one sequence design having a desired prediction of the expression of a gene within the plant cell, and modifying an expression control region to conform to the at least one sequence design within the plant cell in order to obtain a modified plant cell having the desired expression of the gene.

A fifth aspect of the invention provides for a modified plant cell obtained by, or obtainable by, the methods described herein.

A sixth aspect of the invention provides for a modified plant derived from a plant cell as described herein.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is a graph showing the results of an input differential expression analysis (DESeq2) experiment in potato (vr. Georgina) tuber, composed of two bulk RNA-seq datasets, collected at 0 hours and 36 hours post-induction of tuber bruising. Wild type PPO2 has been observed to have 7.5 log 2fold expression increase after 36 hours versus 0 hours of the tuber being exposed to bruising damage. PPO2 has been found in top 3% of the most upregulated genes.

FIG. 2 is a representation of a phylogenetic tree depicting sequence similarity relationships between the 74,000 promoters predicted to be downregulating PPO2 expression. The differential expression for evolved PPO2 sequences is predicted to be a log 2fold−11 in comparison to wild type PPO2 expression in a non-bruised potato. The overall predicted differential expression change is log 2fold−18.5 when compared to wild type PPO2 expression in a potato with induced bruise damage for 36 hours.

FIG. 3 is a map of the 6034bp plasmid (PHY023) used for transient transfection of A. thaliana protoplast plant cells. The plasmid vector backbone with variable nMYB113 promoter regions was used to assemble the plasmid pool for in vivo library validation.

FIGS. 4(a)-4(c) show results of a fluorescence activated cell sorting (FACS) analysis of A. thaliana protoplast leaf cells and demonstrates higher green fluorescence in a population of cells transiently transfected with a synthetic designed nMYB113 promoter library set (b) according to one embodiment of the invention when compared to the wild type MYB113 promoter (a). The transfection positive control is shown in (c). The axes are: x-axis level of green fluorescence, y-axis level of red fluorescence. The axes are in log 10 scale. Red fluorescence is observed on successful transient transfection of A. thaliana protoplasts.

FIG. 5 is a graph of the results of a transcriptional activity assay for a subset of the nMYB113 promoter library tested by transient transfection in A. thaliana protoplast leaf cells and SuRE-Seq. The solid line point indicates threshold activity for the wild type MYB113 promoter, and the dots indicate the transcriptional profile of a subset of 304 variants of a nMYB113 library. The x-axis shows the median log 2fold differential expression change in comparison to the wild type MYB113 promoter and the y-axis shows the statistical metric p-value in −log 10. All the variants above the threshold line have statistically different expression pattern to wild type MYB113 with a p<0.05.

FIG. 6 shows a histogram of 23 nMYB113 promoter variants identified from a nMYB113 library to have significantly (p<0.05) higher gene expression measured by SuRE-seq than the wild type promoter. The y-axis shows the median log 2fold differential expression change in comparison to the wild type MYB113 promoter. Unique molecular identifiers (UMIs) were used to identify the individual elements within the library.

FIG. 7 shows a graph that demonstrates the expression distribution of one of the nMYB113 elements selected, termed “OP625_short_225” after the SuRE-seq with UMI barcodes. The distribution was made using multiple different UMIs. The x-axis indicates log 2fold change in expression with the axis normalised based on the wild type MYB113 promoter distribution. The y-axis shows the density of different UMIs for each element.

FIGS. 8(a)-8(c) show bright field and fluorescence micrographs of transiently transfected A. thaliana protoplast leaf cells. The images show (a) expression of GFP reporter and 35S-mCherry control for a wild type MYB113 promoter; (b) reporter expression with an individual nMYB113 promoter variant A2.1DEL; (c) shows a positive control. The fluorescence output of the nMYB113 promoter variant A2.1DEL can be qualitatively compared to wild type MYB113 promoter output as well as to the transgenic promoter 35S.

FIG. 9 shows a CLUSTALW sequence alignment for a novel PPO2 promoter variant (SEQ ID NO: 1) compared to wild type sequence (SEQ ID NO: 2).

FIG. 10 shows a CLUSTALW sequence alignment for a novel promoter variant (OP625_225) compared to the wild type MYB113 promoter sequence.

FIGS. 11(a)-11(b) show graphs that demonstrate the predictive capability of a machine learning (ML) model according to an embodiment of the present invention in data sets taken from wheat Triticum aestivum. (a) shows the natural variation of RNA-Seq FPKM reads, (b) shows the ML prediction.

FIGS. 12(a)-12(b) show graphs of model performance for root tissue in wheat, (a) provides the data for the experimental RNA-seq, (b) provides the predictive model data.

FIGS. 13(a)-13(b) show graphs that demonstrate the predictive capability of a machine learning (ML) model according to an embodiment of the present invention in data sets taken from maize. (a) shows the natural variation of RNA-Seq FPKM reads, (b) shows the ML prediction.

FIGS. 14(a)-14(b) show graphs that demonstrate the predictive capability of a machine learning (ML) model according to an embodiment of the present invention in data sets taken from soybean. (a) shows the natural variation of RNA-Seq FPKM reads, (b) shows the ML prediction.

### DETAILED DESCRIPTION OF THE INVENTION

Prior to setting forth the invention, a number of definitions are provided that will assist in the understanding of the invention.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, NY; Ausubel, F. M. et al. (Current Protocols in Molecular Biology, John Wiley & Sons, Online ISSN: 1934-3647); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press; Synthetic Biology, Part A, Methods in Enzymology, Edited by Chris Voigt, Volume 497, pages 2-662 (2011); Synthetic Biology, Part B, Computer Aided Design and DNA Assembly, Methods in Enzymology, Edited by Christopher Voigt, Volume 498, Pages 2-500 (2011); RNA Interference, Methods in Enzymology, David R. Engelke, and John J. Rossi, Volume 392, Pages 1-454 (2005). All references cited herein are incorporated by reference in their entirety. Unless otherwise defined, all technical and scientific terms used herein have the same meaning as commonly understood by one of ordinary skill in the art to which this invention belongs.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

The term ‘expression vector’ is used to denote a DNA molecule that is either linear or circular, into which another DNA sequence fragment of appropriate size can be integrated. Such DNA fragment(s) can include additional segments that provide for transcription of a gene encoded by the DNA sequence fragment. The additional segments can include and are not limited to: promoters, transcription terminators, enhancers, internal ribosome entry sites, untranslated regions, polyadenylation signals, selectable markers, origins of replication and such like. In embodiments of the invention, the DNA sequence fragment may comprise plant expression control sequences, including novel non-wild type plant promoter, enhancer or silencer elements. Expression vectors are typically derived from plasmids, cosmids, viral vectors and yeast artificial chromosomes; vectors are suitably recombinant molecules containing DNA sequences from several sources. Nucleic acid expression and reporter libraries of the types described herein may be comprised within expression vectors.

The term ‘operably linked’, when applied to DNA sequences, for example in an expression vector or recombinantly modified gene construct, indicates that the sequences are arranged so that they function cooperatively in order to achieve their intended purposes, e.g. an expression control sequence such as a promoter sequence allows for initiation of transcription that proceeds through a linked coding sequence as far as a termination sequence.

A ‘polynucleotide’ is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Polynucleotides include DNA and RNA, and may be manufactured synthetically in vitro or isolated from natural sources. Sizes of polynucleotides are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides”.

A ‘polypeptide’ is a polymer of amino acid residues joined by peptide bonds, whether produced naturally or in vitro by synthetic means. Polypeptide of less than around 12 amino acid residues in length is typically referred to as a “peptide”. The term “polypeptide” as used herein denotes the product of a naturally occurring polypeptide, precursor form or proprotein. Polypeptides also undergo maturation or post-translational modification processes that may include, but are not limited to: glycosylation, proteolytic cleavage, lipidization, signal peptide cleavage, propeptide cleavage, phosphorylation, and such like. A “protein” is a macromolecule comprising one or more polypeptide chains.

The term ‘promoter’ as used herein denotes a genetic regulatory element in a DNA sequence to which an RNA polymerase will bind and initiate transcription of the DNA. Promoters play a crucial role in gene expression by providing a binding site for RNA polymerases. When RNA polymerase binds to the promoter region, it initiates the process of transcription. Promoters are typically, but not always, located in the 5′ non-coding regions of genes. The 5′ region refers to the upstream region of a gene, meaning it precedes the actual coding sequence of the gene often denoted by an ATG start codon (e.g. prior to the first exon). Non-coding regions are segments of DNA that do not directly contribute to the formation of a polypeptide or other gene product. These regions can contain various regulatory elements, including the promoter. The primary function of a promoter sequence is to provide a recognition site for RNA polymerase and other transcriptional regulatory proteins, allowing them to interact with the DNA and initiate the transcription process. The binding of RNA polymerase to the promoter region marks the starting point for the assembly of the transcriptional machinery, which ultimately leads to the synthesis of an RNA molecule known as the primary transcript or pre-mRNA. Consequently, promoters are highly diverse in terms of their sequence and structure. They contain specific DNA motifs and sequences that are recognized by transcription factors that further regulate gene expression. Transcription factors can either enhance or inhibit the binding of RNA polymerase to the promoter, thereby influencing the level of gene transcription, often in a cell-type or tissue specific manner.

The term ‘enhancer’ as used herein denotes a genetic regulatory element in a DNA sequence that, when bound by one or more transcription factors, enhances the transcription of an associated gene. Enhancers play a pivotal role in gene expression by regulating the transcription of an associated gene or set of genes within a locus. When an enhancer is bound by one or more transcription factors, it enhances the rate of transcription. Enhancers are typically located at varying distances from the gene(s) they regulate. They can be found either upstream (upstream enhancers) or downstream (downstream enhancers) of the gene(s), and sometimes even within introns within the gene itself. Unlike promoters, enhancers are not necessarily orientation-specific and can function regardless of their orientation relative to the gene. A key function of an enhancer is to provide a binding site for transcription factors and regulatory complexes. When specific transcription factors recognize and bind to the enhancer, they can facilitate the assembly of the transcriptional machinery at the promoter region of the associated gene. This recruitment and interaction of transcription factors at the enhancer and promoter regions enable efficient initiation and regulation of gene transcription. Enhancers exhibit remarkable flexibility and can act over long distances. They can interact with the promoter region of the target gene through three-dimensional looping of the DNA, bringing the regulatory elements into close proximity. This spatial arrangement allows the enhancer-bound transcription factors to directly interact with the transcriptional machinery at the promoter, leading to enhanced transcriptional activity. Enhancers can also possess cell type-specific or developmental stage-specific activity. This means that an enhancer may only be active in certain cell types or during specific stages of development, contributing to the precise regulation of gene expression. The specificity and activity of enhancers are governed by the combination of transcription factors that bind to them, creating a complex regulatory network that determines the timing, level, and specificity of gene expression. Additionally, enhancers can act synergistically with other enhancers or regulatory elements in a combinatorial manner. This cooperation between multiple enhancers allows for fine-tuning of gene expression patterns and enables cells to respond to a variety of environmental cues and signaling pathways. The combinatorial effects of enhancers provide a robust and dynamic mechanism for gene regulation, ensuring the proper functioning and adaptation of cells in different contexts, particularly when imparting tissue specificity in the form of phenotypic gene expression.

The term ‘silencer’ as used herein denotes a genetic regulatory element in a DNA sequence that reduces transcription from an associated promoter; typically they are the repressive counterparts of an enhancer. Silencers play a crucial role in reducing or repressing the transcriptional activity of an associated or adjacent promoter and contribute to the fine-tuning of gene expression. Silencers are typically located in proximity to the promoter region of the gene(s) they regulate. They can be found upstream (upstream silencers), downstream (downstream silencers), or even within introns of the gene. Like enhancers, silencers are not necessarily orientation-specific and can function regardless of their orientation relative to the gene. The main function of a silencer is to provide binding sites for transcription factors that have a repressive effect on gene transcription. When specific transcription factors recognize and bind to the silencer, they recruit co-repressor proteins or inhibit the binding of activator proteins to the promoter region. This interference leads to the repression of transcriptional activity from the associated promoter. Silencers can exert their repressive effects in multiple ways. They can directly interact with the transcriptional machinery at the promoter region, preventing the assembly of the necessary components for transcription initiation. Silencers can also induce chromatin modifications, such as the addition of methyl groups to DNA or the removal of acetyl groups from histones. These modifications alter the chromatin structure, making the DNA less accessible to the transcriptional machinery and inhibiting gene expression. Similar to enhancers, silencers can exhibit cell type-specific or developmental stage-specific activity. This means that silencers may only be active in certain cell types or during specific stages of development, adding another layer of complexity to gene regulation. The specific combination of transcription factors binding to the silencer determines its activity and repressive effect on gene transcription. Silencers can also function in a cooperative manner, interacting with other regulatory elements, such as other silencers or enhancers, to modulate gene expression. By working together, these elements fine-tune transcriptional activity and establish precise gene expression patterns in response to various signals and environmental cues. Hence, through the recruitment of repressive transcription factors and chromatin modifications, silencers function as dampeners of transcriptional activity, allowing cells to precisely regulate gene expression levels. Their cell type-specific and cooperative nature adds complexity to the gene regulatory network and ensures proper gene expression patterns during development and in response to different cellular contexts. In certain contexts a silencer may also be a bifunctional regulatory element that can also act as an enhancer, again depending upon cellular context.

As used herein, the terms ‘3′’ (‘3 prime’) and ‘5′’(‘5 prime’) take their usual meanings in the art, i.e. to distinguish the ends or directionality within polynucleotide sequences. A polynucleotide has a 5′ and a 3′ end and polynucleotide sequences are conventionally written in a 5′ to 3′ direction. The 5′ end is suitably considered to be upstream of the 3′ end of a polynucleotide sequence. Hence, sequence referred to as upstream of a given reference point in a gene, such as the transcription start codon of an open reading frame (ORF), is sequence that is 5′ to the reference point. Likewise sequence denoted as downstream is 3′ to the reference point.

According to the present invention, homology to any of the nucleic acid sequences, such as the expression control sequences described herein, is not limited simply to 100%, 99%, 98%, 97%, 95% or even 90% sequence identity. Many nucleic acid sequences can demonstrate biochemical equivalence to each other despite having apparently low sequence identity. In the present invention homologous nucleic acid sequences are considered to be those that will hybridize to each other under conditions of low stringency (Sambrook J. et al, Molecular Cloning: a Laboratory Manual, Cold Spring Harbor Press, Cold Spring Harbor, NY). However, it may be desired in some cases to distinguish between two sequences which can hybridize to each other but contain some mismatches—an “inexact match”, “imperfect match”, or “inexact complementarity”—and two sequences which can hybridize to each other with no mismatches—an “exact match”, “perfect match”, or “exact complementarity”. Further, possible degrees of mismatch are considered.

The present invention, therefore, provides a novel discovery platform for plant expression control sequences, including novel plant promoter, enhancer and/or silencer elements, that are obtained beyond natural diversity. The present invention utilizes an approach that in embodiments provides a combination of artificial intelligence (AI) techniques as well as the further option of in vivo high-throughput library screening for precise control of plant gene expression. In particular embodiments of the invention, a novel nucleic sequence library of gene expression control elements is provided that may be screened for desired gene expression elements. Plant cells, plant tissue and plants (species, varieties or cultivars) may be generated that comprises one or more novel gene expression elements, allowing for desired traits to be enhanced.

Embodiments of the invention utilize an AI-enabled bioinformatics approach to direct the evolution of novel genetic expression control sequences. An exemplary embodiment of the invention is described in more detail below.

Unless already assembled, a novel DNA genome assembly takes place for a plant subject, for example a plant species or variety of interest, and any related varieties. The working data for genome assembly may comprise a short Illumina reads as well as long reads, or a mixture of long and short reads (e.g. using PacBio or Oxford Nanopore sequencing), and includes bioinformatic assembly methods that take into account plant polyploidy and allelic variance. For example, using a Bayesian statistical framework in a haplotype-based variant detector such as FreeBayes, see Garrison E, Marth G. Haplotype-based variant detection from short-read sequencing. arXiv preprint arXiv: 1207.3907 [g-bio. GN] 2012.

A sequence is identified for the region around/on each annotated coding gene identified within the assembled genome. Coding genes may be identified using reference genomic data, published literature supplementation or homology analysis of the assembled genome. These genomic DNA sequences define individual datastrings which are anchored around and extend up to approximately 100,000 base pairs 5′ upstream from a notional translation initiator location (e.g. a start codon) or a transcription initiation location (e.g. a TATA box). The promoterome may also or alternatively include a similar distance 3′ downstream, and/or including the coding gene as well. This captures a plurality of DNA sequences that may collectively define a ‘promoterome’ for the plant subject. In alternative embodiments of the invention, the promoterome may comprise exclusively sequences that are located 5′, or upstream, of an open reading frame. In an alternative embodiment, the promoterome may comprise exclusively sequences that are located 3′, or downstream, of an open reading frame. The promoterome may include one or more exons, one or more introns, one or more transposable elements, and or one or more heterologous inserted elements that may have been introduced by previous gene editing or recombinant techniques.

In specific embodiments of the invention, the promoterome may comprise a plurality of nucleic acid sequences that extend upstream and/or downstream of a first translation initiation location by up to 100,000 base pairs (bps), 90,000 bps, 80,000 bps, 70,000 bps, 60,000 bps, 50,000 bps, 40,000 bps, 30,000 bps, 20,000 bps, 10,000 bps and 5,000 bps. In an embodiment of the invention, the promoterome is defined as comprising a plurality of sequences extending upstream by a distance that is greater than the distance it extends downstream from a reference point in gene, suitably by a factor of at least 1.5, at least 2, at least 3 and at most at least 4 times. In another alternative embodiment of the invention, the promoterome is defined as comprising a plurality of sequences extending upstream by a distance that is smaller than the distance it extends downstream from a reference point in gene, suitably by a factor of at least 1.5, at least 2, at least 3 and at most at least 4 times. As mentioned previously, the reference point may be a transcription or a translation initiation site, suitably where alternative variants or transcripts may exist for a given gene it may comprise any one of the possible transcription initiation sites.

Transcriptome information may be provided in the form of expression data for the plant subject. The transcriptome may be obtained for a particular plant cell, plant cell type, plant tissue type (e.g. foliage or fruit). A transcriptome can be obtained from bulk RNA sequencing techniques (RNA-seq), as well as UMI (unique molecular identifier or “barcode”) based single cell RNA-seq, for example but not limited to drop-RNAseq, and other RNA-seq techniques which leverage transient transfection such as the STARR-seq and SuRE-seq methods. Also, in certain embodiments of the invention, data inputs may be supplemented with add-on datasets such that assess chromatin accessibility and transcription factor binding information.

Suitable chromatin based sequencing methodologies may include:
ATAC-seq (Assay for Transposase-Accessible Chromatin with high-throughput sequencing): ATAC-Seq is a method to investigate chromatin accessibility in a sample. In this assay the genome is treated with a transposase (enzyme) called Tn5. Tn5 marks open chromatin regions by cutting and inserting adapter sequences which can then be detected by later sequencing. ATAC-seq shows utility in assessing changes in genome wide chromatin accessibility post editing event (Buenrostro et al. Curr Protoc Mol Biol. (2015); 2015:21.29.1-21.29.9).
Chromosome conformation capture (3C) methodologies such as Hi-C analysis may be used to assess chromatin accessibility, 3D organization of the genome and interconnectivity to identify any changes to chromatin accessibility and 3D architecture of the genome including the local chromosome neighbourhood and/or transcription factories (Lieberman-Aiden et al. Science. 2009 Oct. 9; 326(5950): 289-293)
Chromatin immunoprecipitation followed by sequencing (ChIP-seq): ChIP-seq is a central method in epigenomic research. Genome-wide analysis of histone modifications, such as enhancer analysis and genome-wide chromatin state annotation, enables systematic analysis of how the epigenomic landscape contributes to cell identity, development, lineage specification, and disease. In particular, post edit impact on histone modifications should be assessed such as, but not limited to, regulatory elements (H3K27Ac, H3K4Me1), promoter accessibility (H3K4Me3), formation of heterochromatin (H3K9Me3), gene bodies (H3K36Me3, H3K27Me3)—see Furey (2012) Nat. Rev. Genet., 13 (12), pp. 840-852.
Methyl-sequencing (Methyl-seq): This approach assesses the impact of an edit on DNA methylation profiles within the genome thereby estimating changes on chromatin accessibility. Methyl-seq can be carried out using chemical (bisulphite sequencing) or enzymatic approaches (EM-Seq)—see Vaisvila et al. (2021) Genome Res. July; 31(7): 1280-1289.
DNase accessibility data: A deoxyribonuclease (DNase, for short) is an enzyme that catalyses the hydrolytic cleavage of phosphodiester linkages in the DNA backbone, thus degrading DNA. Deoxyribonucleases are one type of nuclease, a generic term for enzymes capable of hydrolysing phosphodiester bonds that link nucleotides. DNase activity is one way to assess chromatin accessibility and to define the importance of a cell-type specific region within the plant tissue/cell of interest.

The analysis of the data obtained from one, or more than one, of the above sequencing techniques allows for the assessment of the promoterome at an epigenetic and transcriptional level. This assessment comprises determination of the activity and chromosomal accessibility of, for example, transcription factor binding sites, transcription factor coding regions, genetic regulatory elements (such as enhancers, silencers, and repressors), DNA methylation, histone modifications, and transcription factories.

Analysis of the promoterome and transcriptome input data is achieved by using sequence-based modelling algorithms that are configured to provide a prediction value of the level of expression of each protein coding gene comprised within the transcriptome and linking the prediction value to a corresponding sequence for an expression control region comprised within the promoterome.

Bioinformatic processing of the expression data may include but is not limited to techniques such as:
Mapping of next generation sequencing reads to create read counts—e.g. HISAT2
Normalisation and expression analysis—e.g. DESeq2
Batch effect removal—e.g. Combat

The sequence-based modelling algorithms may utilise an artificial intelligence (AI) and machine learning (ML) approach. The model may be trained using input data relating to the transcriptomics and promoteromics described previously in order to provide a prediction, termed the prediction value, of the expression of a gene comprised within the genome assembly. The prediction value may be assigned to the gene allowing interrogation of every identified gene within a database of said genes. The database may be interrogated based upon a number of criteria and meta data assigned to each entry, such as the gene sequence, the coding sequence, protein sequence, expression levels, associated UMI, as well as the prediction value. The gene sequence may include the regulatory region (promoter and non-coding parts), as well as other portions of the gene e.g. 5′ and 3′ untranslated regions (UTR), coding sequence (CDS), introns, exons and others regions. In addition, the models may be used to interrogate the expression value of a new sequence, outside the database of genes, with further transcriptional and in vivo validation performed subsequently if desired.

As used herein, the term “prediction value” may represent a number or score that characterises the expression of a gene. The prediction value may be a relative value, such as the level of expression—positive or negative—relative to a notional benchmark, such as the expression of a housekeeping or other appropriate reference gene. As such, the prediction value may be a number, a log value or some other nondimensional value—e.g. a colour or alphanumeric character coding where the prediction value falls between specific threshold bandings. In a alternative embodiment, the prediction value may be an absolute value that corresponds to a predicted quantification of gene expression as typically determined via one or more techniques such as, but not limited to, RNA sequencing (RNA-seq), microarray analysis, quantitative RT-PCR (qPCR), Northern or Western blotting, in situ hybridization, and immunohistochemistry. Hence, according to a specific embodiment of the invention, the prediction value is represented as the RNA-seq count for a given sequence design. The RNA-seq count may be provided as in unit counts such as FPKM (fragments per kilobase of transcript per million) or TPM (transcript per million).

Sequence-based modelling architectures and sequence prediction algorithms, including those common to natural language processing, may be used in embodiments of the invention that provide a prediction value of the level of expression of each protein coding gene comprised within the transcriptome and linking the prediction value to a corresponding sequence for an expression control region comprised within the promoterome. Suitable sequence-based modelling algorithms may include artificial neural networks (ANNs), for example, such as convolutional neural networks (CNN), recurrent neural networks (RNN), including bidirectional RNNs, transformers and masked language models. These algorithms are specific realisations of a family of machine learning models called neural networks, where the network “neurones” are arranged in an architecture that is amenable to modelling of sequential data, such as genomic sequences. They have been used in a variety of biological applications known to the skilled person. A description of the algorithms and their successful applications to biology is provided in, for example, Greener et al. Nature Reviews Molecular Cell Biology, volume 23:40-55 (2022).

According to an embodiment of the invention, a plurality of non-wild type sequence designs are generated for novel expression control sequences that are most likely to provide a desired expression profile for an operably linked coding sequence. The plurality of non-wild type sequence designs are informed by the prediction value generated by the sequence-based modelling steps described herein. Hence, where it is desirable to increase expression of an operably linked coding sequence, a sequence exhibiting a predicted high level of expression value is sought. Conversely, where it is desirable to decrease expression of an operably linked coding sequence, a sequence exhibiting a predicted low level of expression value is sought.

The plurality of sequence designs for novel expression control sequences are generated via an in silico mutagenesis approach. Such approaches may include rule based sequence and/or generative sequence designs. In specific embodiments of the invention a ‘minimal change’ specification is adopted which favours a minimal amount of basepair alterations, insertions or deletions compared to a template/starting wildtype (WT) promoter. A crop specific requirement of ‘minimal change’ can address concerns with the use of novel breeding technologies (NBTs) such as CRISPR-CAS guided endonuclease based genome editing to create non-transgenic, targeted changes to crop genomes. In some jurisdictions such approaches are classed as a non-genetically modified organisms (non-GMOs).

The minimal change approach is also a major point advantage in comparison to other known synthetic AI based design approaches used in industrial biotechnology or medicine that tend to optimise attributes e.g. ‘fitness’ to a local or global maximum without any regard for the number of changes in comparison with a given starting sequence. In contrast, a minimal change approach allows for attribute optimisation, e.g. fitness, that passes a quality control (QC) threshold for in planta performance but considers (a) promoter/enhancer/silencer's native function and (b) the real-world legislative and the regulatory aspects of DNA changes in plants destined to be used as commercial crops.

Hence, in embodiments of the invention a minimal change threshold is established for non-wild type sequence designs in relation to the number and/or type of alterations permitted to be made for an expression control sequence from the wild type. This minimal change threshold may be incorporated into the rule based sequence and/or generative sequence design algorithms that adopt sequence perturbation to design mutated sequences with desired gene expression profiles. In embodiments of the invention, concepts from the field of explainable AI, such as by assigning feature importance or interaction strength, which may be used to deduce the placement and structure of the alterations in the wild-type sequence that are most likely to achieve the desired change in its expression value prediction (see Molnar, C. “Interpretable Machine Learning: A Guide For Making Black Box Models Explainable” 2^{nd} Edition (2022) ISBN: 979-8411463330).

The novel non-wild type sequence designs may be scored via in silico high throughput techniques, e.g. use of one or more AI based trained models, to determine the top-ranked sequence designs that are most likely to provide the optimal desired expression profiles.

In vivo verification of novel non-wild type sequence designs may be carried out via design and construction of a library of novel genetic expression control sequences created by de novo DNA synthesis. Comparable wild type genetic expression control sequences can be synthesised de novo or amplified from plant subject source material by standard recombinant techniques such as polymerase chain reaction (PCR).

The methods of the present invention may be used to generate a diverse range of genetic control elements that may be introduced into plant cells and/or tissues. Suitably, a plant cell may be selected from a gametophyte, a reproductive cell, a vegetative cell and/or a meristematic cell.

In certain embodiments the plant cell is in the form of a protoplast. As used herein, the term “plant protoplasts” (also referred to simply as “protoplast”, throughout this disclosure) refers to a plant cell that has had its cell wall completely or partially removed. Removal of cell wall can be effected by mechanical, chemical or enzymatic means. In embodiments, protoplasts are obtained from suitable plant material using cell wall digestive enzymes. For example, enzymes such as cellulase, macerozyme, pectinase, hemicellulase, pectolyase, driselase, xylanase and combinations thereof may be suitable for use in the context of the invention. In embodiments, cellulase may be used at a concentration of 1w %-1.5 w %. In embodiments, macerozyme may be used at a concentration of 0.2w %-0.4 w %. In embodiments, hemicellulase may be used at a concentration of 2w %-5 w %. In embodiments, pectolyase may be used at a concentration 0.01w %-0.5 w %. In embodiments, driselase may be used at a concentration of 0.5w %-2w %. Protocols for obtaining protoplasts from plant tissues are known in the art and will not be discussed further here.

In embodiments, suitable plant tissue is selected from: leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, or leaf sheath. In specific embodiments plant cell material may include root tissue, leaf mesophyll and/or cultured callus. In embodiments, the protoplasts are obtained using the protocol described in Yoo, Cho, & Sheen (2007) Nature Protocols volume 2, pages 1565-1572, which is incorporated herein by reference.

In embodiments, the in vivo validation may comprise methods to detect and optionally to select encapsulated protoplasts or plant cells based on one or more desired characteristic. The systems of the invention comprise an optical detection system, such as a fluorescence detection system. In combination with a sorting mechanism, this enables selection of protoplasts or cells having a fluorescent marker indicative of a characteristic of interest or as a simple expression reporter. In embodiments, the methods of the invention comprise introducing the coding sequence for a fluorescent protein into a protoplast or plant cell culture. In such embodiments, the methods of the invention may comprise using a fluorescence detection system, such as a fluorescence activated cell sorting (FACS) system, to detect the expression of a fluorescent protein in the protoplasts or plant cells being assayed. Suitable screening and propagation methodologies are described and exemplified, for example, in WO-A-2020/212713.

Some examples of intracellular reporter proteins that may serve as fluorescent markers of gene expression include green fluorescent protein (GFP) and homologues or derivatives thereof, such as enhanced GFP (eGFP), blue fluorescent protein (BFP, Azurite, mKalama1), cyan fluorescent protein (CFP, CyPet), yellow fluorescent protein (TFP, Citrine) and mCherry. This allows for transfected protoplasts to be readily identified using conventional cell sorting techniques.

In embodiments, a fluorescence detection system may be configured to detect both a signal indicative of the presence of chlorophyll and a signal indicative of the presence of a fluorescent marker indicative of a characteristic of interest. In such embodiments, the detection system may be coupled to a sorting mechanism, and the system may be configured such that the sorting mechanism separates microcapsules between two different channels based on the combined presence of a signal indicative of the presence of chlorophyll and a signal indicative of the presence of a fluorescent marker.

In embodiments, a chemiluminescence detection system may also be utilised. In combination with a sorting mechanism, this enables selection of protoplasts having a chemiluminescent marker indicative of a characteristic of interest. In embodiments, the methods of the invention comprise introducing the coding sequence for one or more chemiluminescent proteins (e.g. luciferin, aequorin, etc.) into a protoplast culture. In such embodiments, the methods of the invention may comprise using a luminescence detection system to detect the expression of a chemiluminescent protein in the encapsulated protoplasts. Protoplasts, as explained above single encapsulated protoplasts may be manually or automatically placed into a tissue culture system, for example on an agar gel plate containing plant growth media or into a micro well plate containing gel and liquid with plant growth promoting media.

In embodiments, a callus inducing media may be used. For example, media such as Gamborg B5, Murashige Skoog or others may be used. As will be understood by the skilled person various salts, vitamins and auxins, cytokinins and/or other hormones promoting growth of the single protoplast into a callus may be included in the medium, or the tissue culture plate.

In embodiments, calli having achieved a predetermined size may be moved into plant growth media with different auxins and cytokinins ratio to induce shoot formation. In embodiments, calli having undergone shoot formation may be moved to yet another plant growth medium to induce root formation. Preferably, any manipulation of the calli may be done under sterile conditions.

Small plantlets resulting from the above process may then be used in conventional micro propagation techniques.

According to embodiments of the invention, methods and apparatus may be provided for efficiently engineering and recovering plants or propagatable plant material (e.g. seeds) comprising a non-wild type gene expression pattern or signature of interest. The ability to utilise of single cell rapid phenotyping of a novel library and high efficiency of recovery of whole plants that are afforded by the invention are particularly advantageous in the context of plant genetic engineering.

The target plant indicated or desired for analysis utilising the methods of the present invention is referred to as the “plant” or a “plant subject”. Plant subject species and genera where the invention is envisioned to be of particular use include, but are not limited to, Solanum spp. (e.g. S. lycopersicum, S. tuberosum, S. melongena, S. muricatum, S. betaceum); Brassica spp. (e.g. B. oleracea, B. napobrassica, B. napus, B. cretica, B. rupestris and B. rapa); Capsicum spp. (e.g. C. annuum, C. baccatum, C. chinense, C. frutescens, C. pubescens); Lupinus spp. (e.g. L. angustifolius, L. albus, L. mutabilis and L. luteus); Phaseolus spp. (e.g. P. acutifolius, P. coccineus, P. lunatus, P. vulgaris and P. dumosus); Vigna spp (e.g. V. aconitifolia, V. angularis, V. mungo, V. radiata, V. subterranea and V. unguiculata); Vicia faba; Cicer arietinum, Pisum sativum, Lathyrus spp. (e.g. L. sativus and L. tuberosus); Lens spp. (e.g. L. culinaris and L. esculenta); Glycine max; Psophocarpus; Cajanus cajan; Arachis hypogaea; Lactuca spp. (e.g. L. sativa, L. serriola, L. saligna, L. virosa and L. taterica); Asparagus officinalis; Apium graveolens; Allium spp. (e.g. A. cepa, A. oschaninii, A. ampeloprasum, A. wakegi, A. porrum, A. sativum and A. schoenoprasum); Beta vulgaris; Cichorium intybus; Taraxacum officinale, Eruca spp. (e.g. E. vesicaria and E. sativa); Cucurbita spp. (e.g. C. argyosperma, C. digitata, C. pepo, C. moschata, C. ecuadorensis, C. ficifolia, C. foetidissima, C. galeottii, C. lundelliana, C. maxima, C. moshata, C. pedatifolia, C. radicans); Spinacia oleracea; Nasturtium officinale; Cucumis spp. (e.g. C. sativus, C. melo, C. hystrix, C. picrocarpus and C. anguria); Olea europaea; Daucus carota; Ipomoea batatas; Ipomoea eriocarpa; Manihot esculenta; Zingiber officinale; Armoracia rusticana; Helianthus spp. (e.g. H. annuus and H. tuberosus); Cannabis spp. (e.g. C. sativa and C. indica); Pastinaca sativa; Raphanus sativus; Curcuma longa; Dioscorea spp. (e.g. D. rotundata, D. alata, D. polystachya, D. bulbifera, D. esculenta, D. dumetorum, D. trifida and D. cayennensis); Piper spp. (e.g. P. aduncum, P. guineense and P. nigrum); Zea spp. (e.g. Z. mays and Z. diploperennis); Hordeum spp. (e.g. H. vulgare, H. pusillum, H. murinum, H. marinum, H. jubatum and H. intercedens); Gossypium spp. (e.g. G. hirsutum, G. barbadense, G. arboreum and G. herbaceum); Triticum spp. (e.g. T. aestivum and T. timopheevii); Vitis vinifera; Prunus sp. (e.g. P. avium, P. armeniaca, P. cerasifera, P. cerasus, P. domestica, P. persica and P. dulcis); Malus domestica; Pyrus spp. (e.g. P. communis, P. cordata and P. pyrifolia); Fragaria vesca and Fragaria x ananassa; Rubus idaeus; Saccharum officinarum; Sorghum saccharatum; Musa balbisiana and Musa x paradisiaca; Oryza sativa; Nicotiana tabacum; Arabidopsis thaliana; Citrus spp. (e.g. C. x aurantiifolia, C. x aurantium, C. x latifolia, C. x limon, C. x limonia, C. x paradisi, C. x sinensis and C. x tangerina); Populus spp. (e.g. P. tremula, P. balsamifera and P. tomentosa); Tulipa gesneriana; Medicago sativa; Abies balsamea; Avena orientalis; Bromus mango; Calendula officinalis; Chrysanthemum balsamita; Dianthus caryophyllus; Eucalyptus spp. (e.g. E. leucoxylon, E. maculata, E. polybractea, E. sargentii); Impatiens biflora; Linum usitatissimum; Lycopersicon esculentum; Mangifera indica; Nelumbo spp. (e.g. N. nucifera and N. pentapatala); Poaceae spp.; Secale cereale; Tagetes erecta; and Tagetes minuta. Plants and plant cells of any of the aforementioned species with modified sequences comprised within their genomes, particularly within one or more expression control sequences, may be generated by the methods described herein.

Novel gene expression control sequences, such as those identified via the methods described herein, may be used in techniques involving targeted gene editing in a plant cell or a plant subject. Various approaches to targeted gene editing are available to the skilled person, including those techniques that rely on sequence guided endonucleases such as CRISPR/Cas-based genome editing systems. Hence, embodiments of the present invention may provide methods for producing a genome edited plant or plant propagatory material (e.g. seeds). CRISPR/Cas-based genome editing systems can be targeted to specific nucleic acid sequences. For example, a guide RNA of a CRISPR/Cas is designed to associate with a nucleic acid molecule such that the Cas endonuclease can recognize a protospacer adjacent motif (PAM) sequence in the nucleic acid molecule and cleave (or nick) the nucleic acid molecule. Guide RNAs (gRNAs) in CRISPR/Cas genome editing systems are targeted to specific locations within the genome of the plant subject that are adjacent or proximate to an appropriate nucleotide protospacer adjacent motif (PAM) sequence, as will be known to the skilled person. The gRNAs may be used to target specific locations within the plant genome that are comprised within or close to a wild type expression control element. Cleavage of the sequence within or close to the gene expression control element may allow for disruption of the endogenous sequence, or for insertion of a new genetic control sequence into the gene. Appropriate novel genetic control sequences will comprise one or more of the validated non-wild type sequence designs identified by the present methods. Highly specific targeting allows CRISPR/Cas-based genome editing technology to undertake minimal genome alterations but to maximum phenotypic effect. Hence, it is believed that such gene editing techniques hold great promise for plant genome engineering because of its simplicity and efficiency.

In a specific embodiment of the invention, a process is provided in which a novel sequence design identified by the methods of the invention described herein is utilised for the modification of a plant cell. The novel sequence design is used to inform selection of a target gene or genomic region, whereupon designing a guide RNA (gRNA) that guides a Cas protein (e.g., Cas9 or Cas12a) to the target site, construction of a CRISPR/Cas system comprising the Cas protein and gRNA, and delivery of the CRISPR/Cas system into plant cells is via established methods (e.g., Agrobacterium-mediated transformation). DNA cleavage occurs at the desired genomic location by the Cas protein followed by activation of the plant cell's DNA repair mechanisms, e.g. via non-homologous end joining (NHEJ) and homology-directed repair (HDR), Subsequent screening and selection of edited plant cells or tissues is carried out. Hence, the method allows for precise modifications that introduce the novel sequence design into the plant genome, e.g. through HDR or disruption of target genes via NHEJ. The edited plant cells or tissues can be regenerated into whole plants, enabling further characterization and evaluation of the genomic modifications and resulting phenotypic effects. The disclosed CRISPR/Cas genome editing method has significant potential for applications in plant research, crop improvement, disease resistance enhancement, and the development of novel plant varieties.

In a specific embodiment of the invention, a system is provided that comprises at least one processor configured to operate a DNA sequence model based on a convolutional neural network and transformer-based architecture. The model is trained to provide a prediction value, in the form of a numerical output, which represents a level of gene expression from DNA sequence information alone. In one embodiment, an input gene is defined as a ˜3000bp sequence centred around a TSS (transcription start site) of the gene. The prediction value is represented as the predicted RNA abundance count, a standard way of representing gene expression, determined by measuring the amount of RNA molecules corresponding to an individual gene at a given timepoint and within a given tissue. The RNA abundance is usually obtained experimentally by performing an RNA-seq assay. In this way RNA is extracted from plant tissue and a sequencing methodology is performed that counts the number of RNA transcripts linked to a gene that are present. The numbers are usually presented in unit counts such as FPKM (fragments per kilobase of transcript per million) or TPM (transcript per million). Using model training with 80% of given species genes as input genes and RNA-seq counts in FPKM or TPM for given conditions can be determined. For example, RNA-seq counts may be determined in a given plant for specific tissue types, such as leaf, or for environmental stress such as drought, In accordance with the methods of the invention it is possible to train the model to predict an RNA-seq count based on an input gene sequence alone, i.e. without having to experimentally measure the RNA-seq count. This surprisingly powerful ability to predict gene expression output from a query DNA sequence alone, enables production of novel expression control sequences, such as by iterative modification, to alter the prediction value either positively (to up-regulate gene expression) or negatively (to down-regulate gene expression).

Embodiments of the present invention allow for the combination of in silico DNA sequence generation/promoter mutagenesis, followed by model prediction (setting of a prediction value), for example, by assigning RNA-seq count to a natural or synthetic query sequence. This approach makes it possible to modulate and/or improve gene expression rapidly in plants, by having an iterative loop with predictive gene expression for promoter and gene sequences, without having to validate each round of sequence changes in the lab. In practice, the sequence designs that are generated and that pass a multistep assessment with a prediction value (e.g. RNA-seq count) from the model may be further validated in the wet lab to further characterize the behaviour in plant cells and full plants. The data generated via wet lab validation may also be used to inform the models further.

The invention will now be further illustrated by way of the following non-limiting examples.

### EXAMPLES

### Example 1—In Silico-Assisted Design of an Altered PPO2 (PGSC0003DMG400018916) Promoter in Potato (Variety Georgina)

Potato bruising is a common agricultural problem, creating significant losses every year. One of the main genes affecting potato bruising is polyphenol oxidase 2 (PPO2). Control of PPO2 expression in potato could lead to a non-bruising potato.

The intention of this example is to evolve the regulatory region of PPO2 gene in a potato (variety Georgina) to down-regulate the expression of PPO2 in potato and minimise discoloration associated with potato bruising.

### Starting Data Inputs:

Whole potato genome sequence (vr. Georgina, HiFi PacBio+Illumina PE150 reads).

Selected 5,000 basepairs (bp) sequence region upstream of 5′UTR of all annotated coding sequence genes known in the potato (vr. Georgina) genome.

A differential expression analysis of the potato transcriptome (vr. Georgina) after 36 hours of exposure to bruising damage versus a non-bruised transcriptome (0 hours).

### Results:

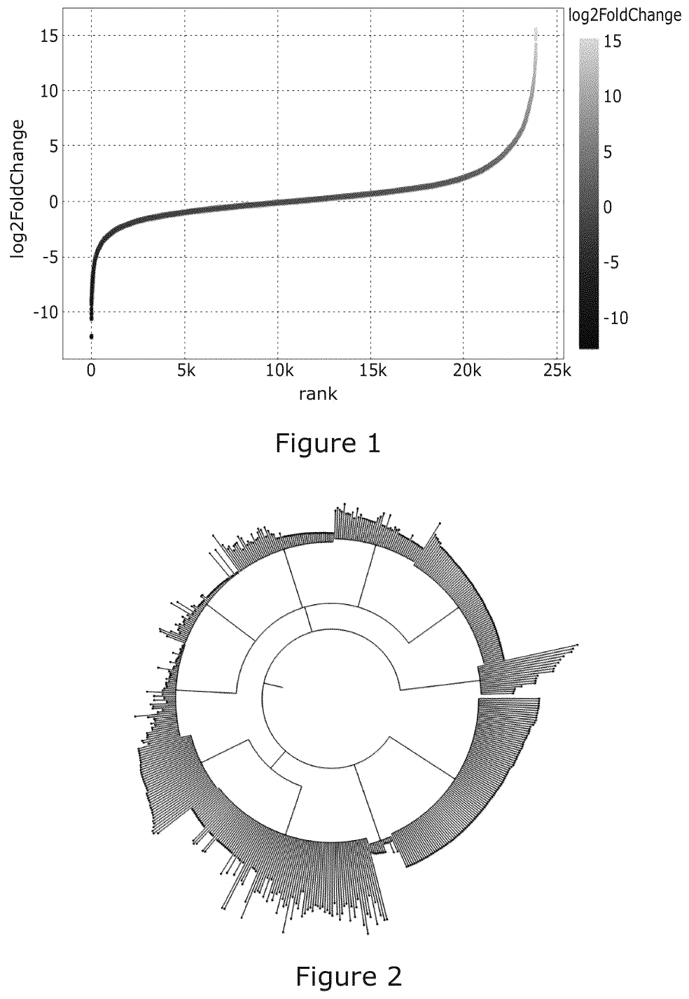

Analysis of the potato transcriptomes identified those genes upregulated in response to tuber bruising (see FIG. 1). Of these the polyphenol oxidase 2 (PPO2) gene was found to be present in the top 3% of upregulated genes. PPO2 was selected as a candidate for further analysis.

19,000,000 promoter variants of 5,000bp from the 5′UTR PPO2 promoter region were screened computationally varying across 917 DNA sites within the potato genome. The maximum amount of the basepairs altered was 150bp, or a maximum of 3%, of the 5000 basepairs selected.

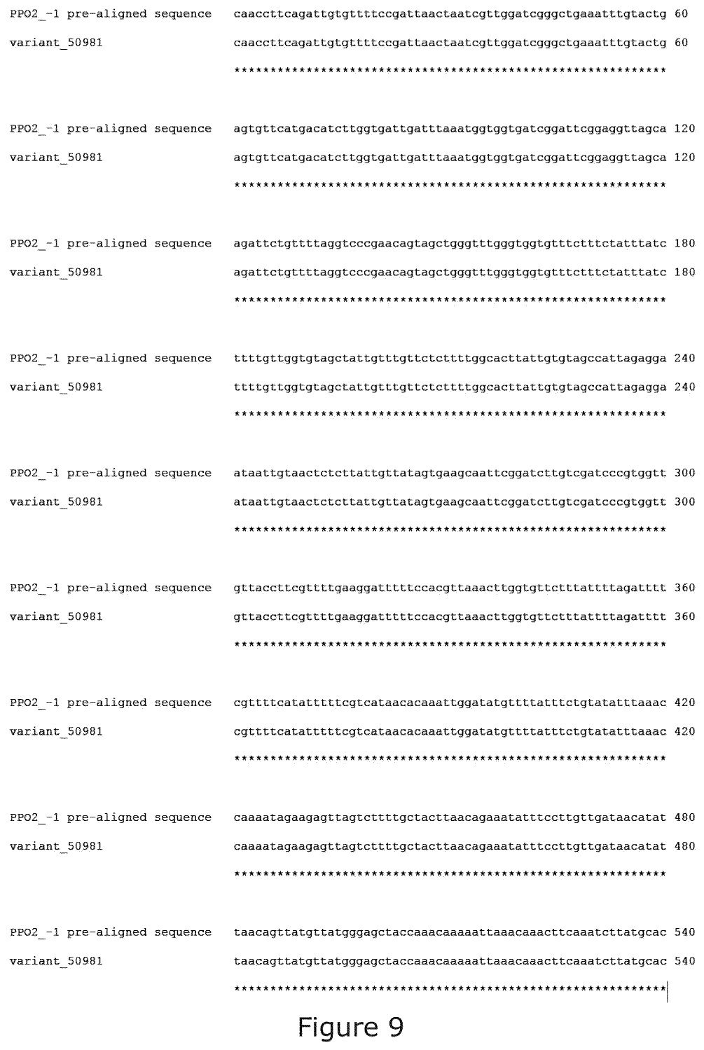

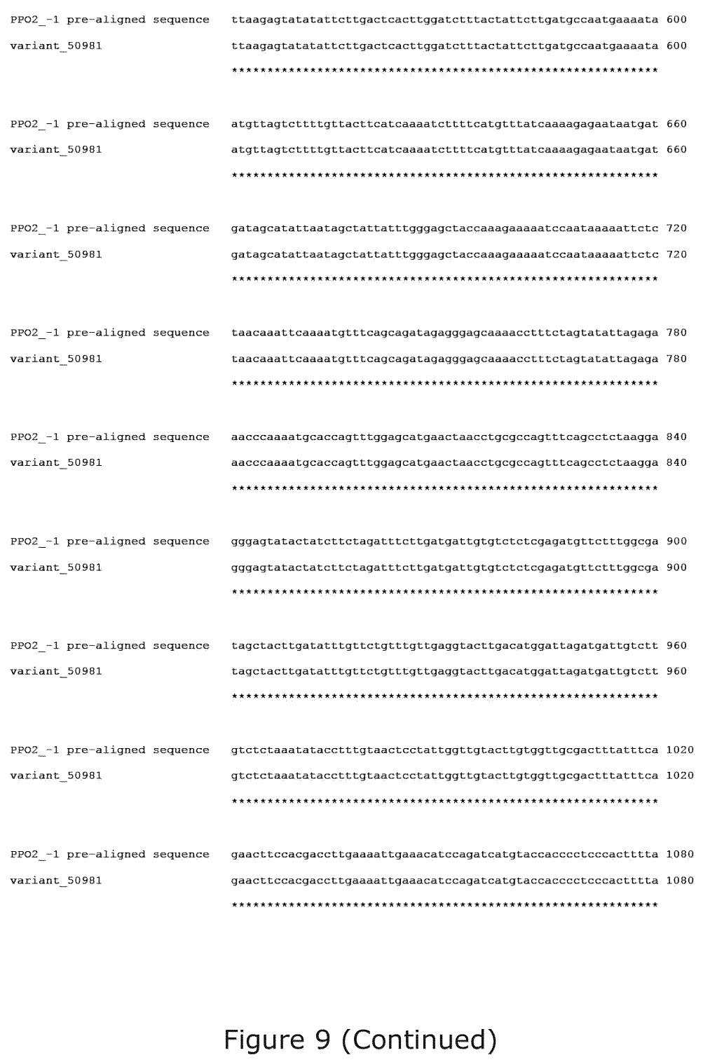

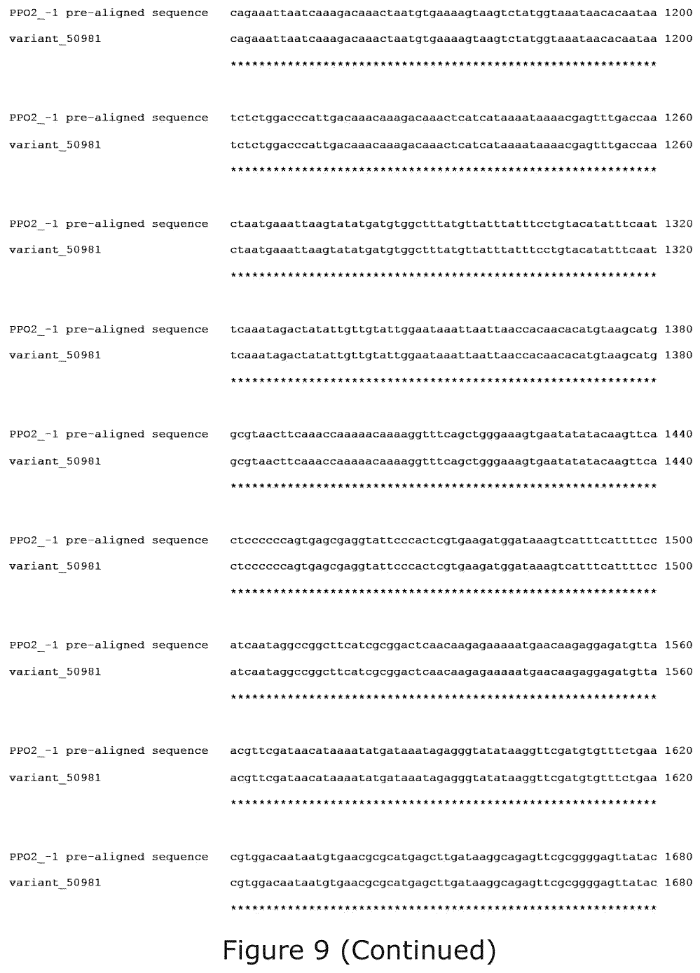

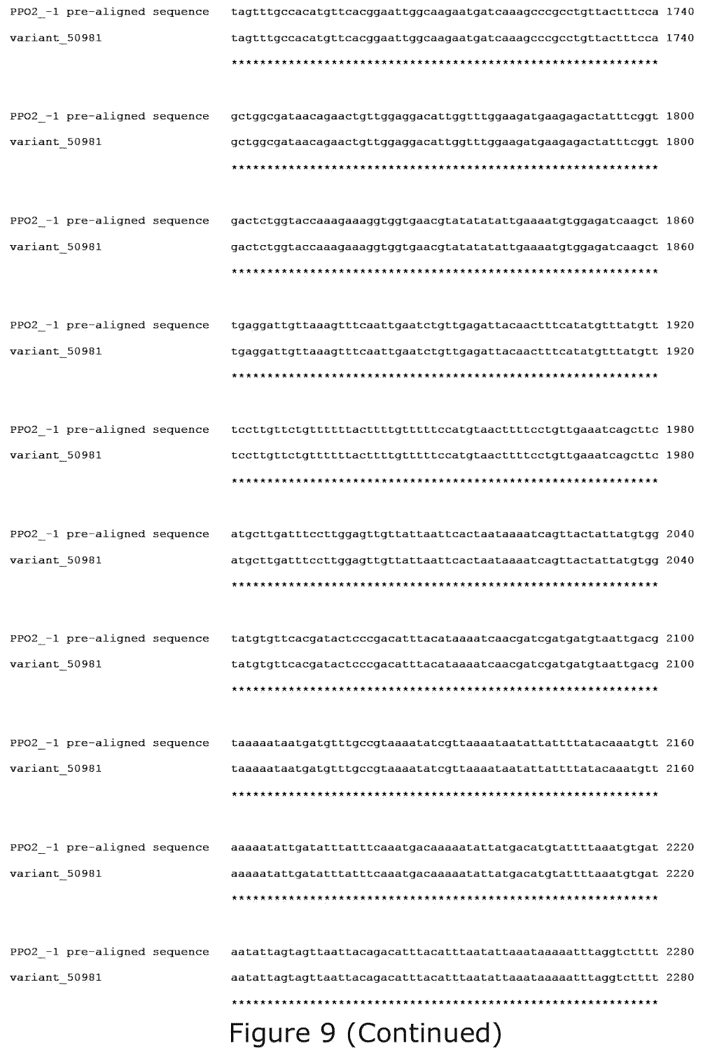

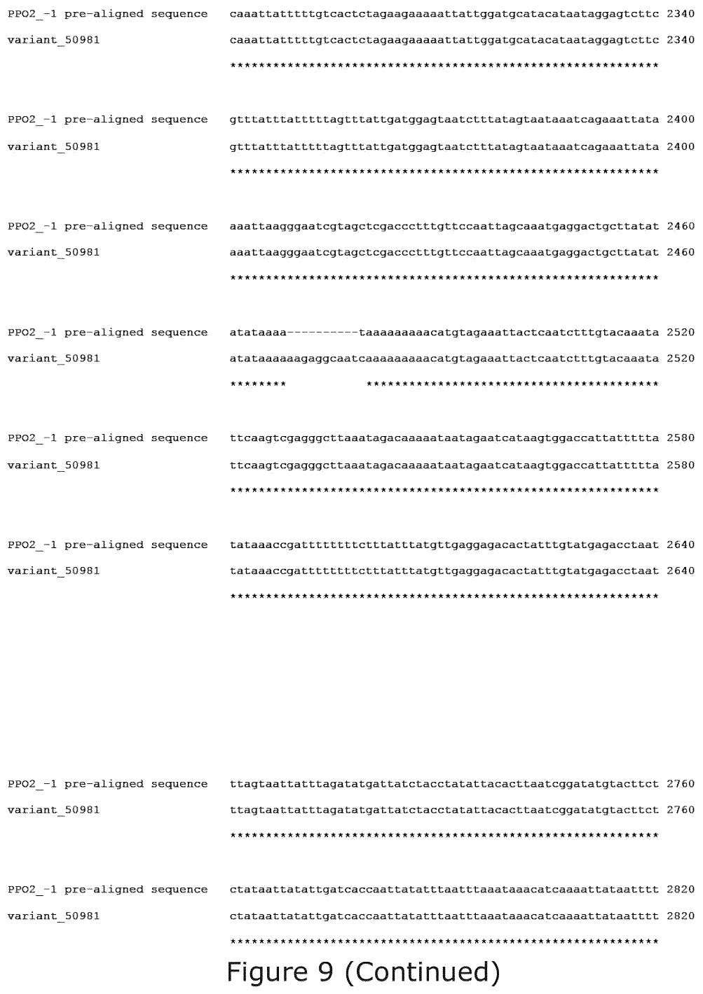

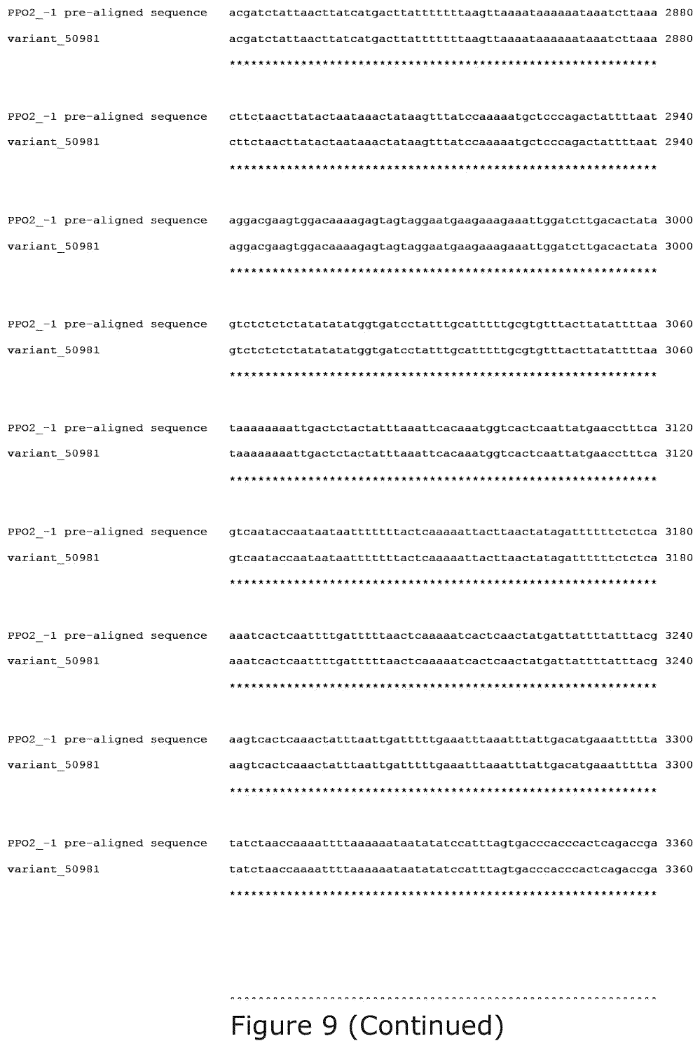

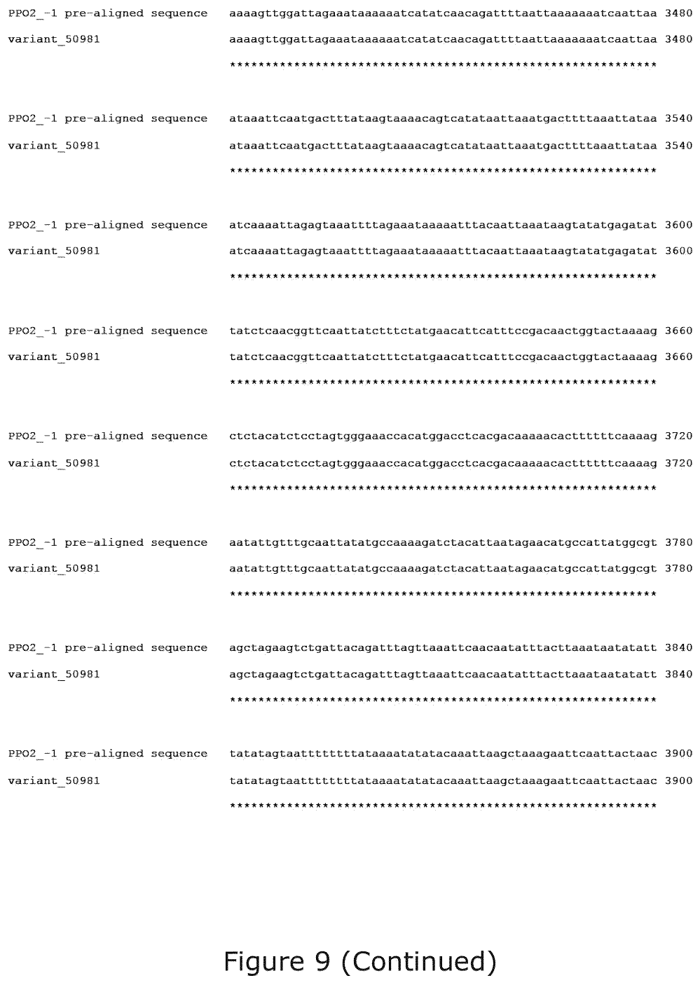

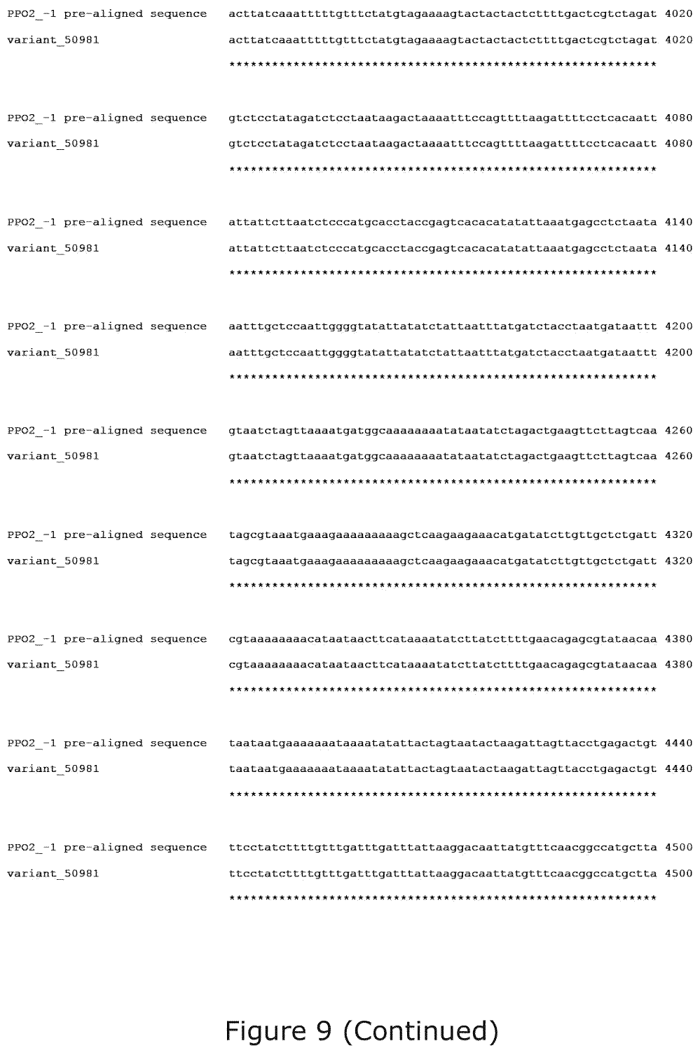

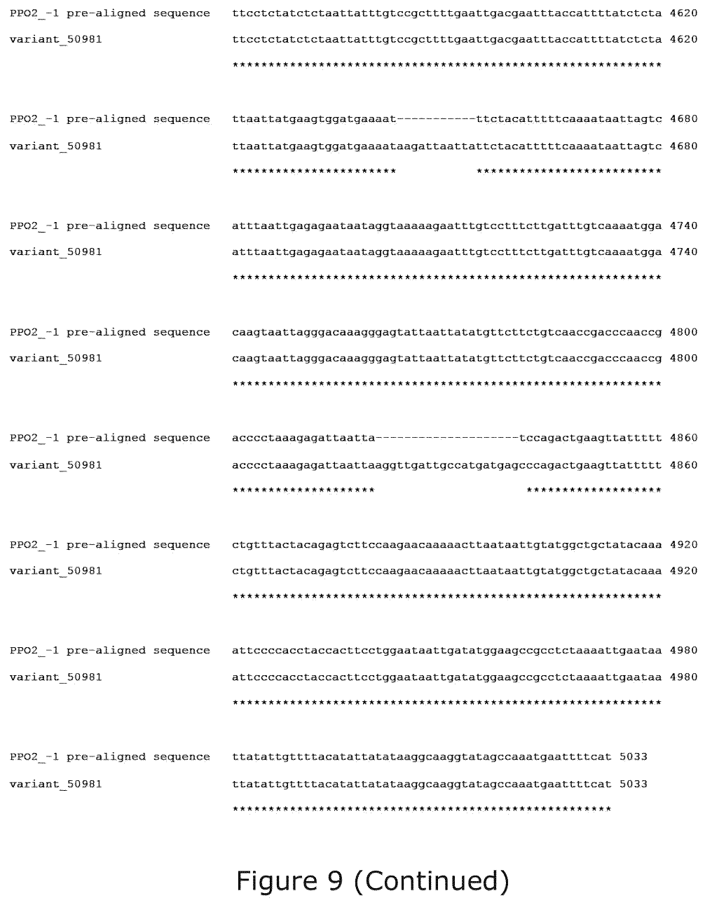

Out of the 19,000,000 promoter variants, using inference with a model trained to classify expression of potato promoterome in the tuber, 74,000 in silico evolved promoter variants have been predicted to significantly downregulate PPO2 with a classed differential expression of −11 log 2fold change for a potato (vr. Georgina) exposed for 36 hours to bruising damage versus a potato that was not exposed (see FIG. 2). An exemplary variant promoter sequence called variant_50981 (SEQ ID NO: 1) is shown aligned to wild type PPO2 (SEQ ID NO: 2) in FIG. 9.

### Example 2—In Silico Assisted Evolution of an Altered MYB113 (AT1G66370) Promoter in Arabidopsis thaliana (Ecotype Col-0)

A. thaliana is a well characterised plant science model organism and here it used for the demonstration of the present approach.

Anthocyanin based purple discoloration is a change A. thaliana undergoes when stressed. It is controlled by a transcription factor gene MYB113, which is very tightly regulated. Under non-stress conditions is not expressed at any physiologically relevant level. By evolving a regulatory region for MYB113 that can drive the expression of MYB113 in the absence of stress, this can generate normally grown plants but with purple leaves.

The objective of this experiment is to use the present in silico assisted evolution algorithms to evolve the regulatory region of MYB113 gene in A. thaliana (ecotype Col-0) to up-regulate the expression of MYB113 in leaves in the absence of stress and thereby to generate plants with purple-coloured leaves.

### Starting Data Inputs:

Arabidopsis thaliana genome (ecotype Col-0, TAIR10 assembly) accessed from Ensembl Plants (see Yates et al., Nucleic Acids Research, Volume 50, Issue D1, 7 Jan. 2022, Pages D996-D1003).

Variable base pair sequence region upstream of the 5′UTR of all annotated coding sequence genes in the A. thaliana (ecotype Col-0) genome.

Transcriptome expression dataset obtained from University of Toronto (Klepikova Atlas) of Arabidopsis (ecotype Col-0) of RNA levels in different plant organs at different developmental ages (Klepikova et al. (2016), The Plant J., Volume 88, Issue 6; 1058-1070).

### Results:

9,000,000 promoter variants of 754bp 5′UTR MYB113 promoter region were screened computationally. The maximum amount of the basepairs (bp) altered was 35bp, or a maximum of 4.6% of the 754 base pairs selected.

Out of the 9,000,000 promoter variants, the model evaluated 16,000 promoter variants predicted to have significantly upregulated MYB113 expression in Arabidopsis leaves compared to wild type MYB113 sequence.

To validate the model prediction, 1250 sequences were chosen arbitrarily (nMYB113) with a concern for ease of DNA library synthesis.

A plasmid pool containing the 1250 variations of a 754bp 5′ regulatory region of MYB113 gene immediately upstream of 5′ MYB113 UTR was assembled using conventional techniques known to the skilled person. Other components of the plasmid included:
1. 5′ MYB113 UTR derived from A. thaliana TAIR10 genome,
2. a coding gene coding for green fluorescent protein (EGFP),
3. a 4 codon variable region at the start of the GFP gene acting as a unique molecular identifier (UMI),
4. 3′ MYB113 UTR derived from A. thaliana TAIR10 genome,
5. 35S derived terminator
6. 35S expression cassette including 35S promoter, 5′UTR, mCherry gene coding for a fluorescent reporter gene, 3′ UTR and terminator.

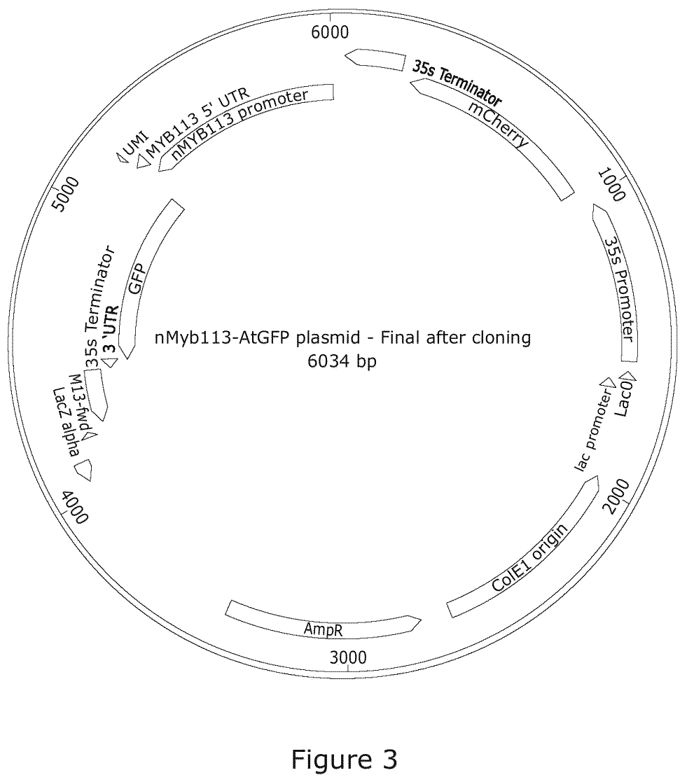

The vector map for the plasmid is shown in FIG. 3.

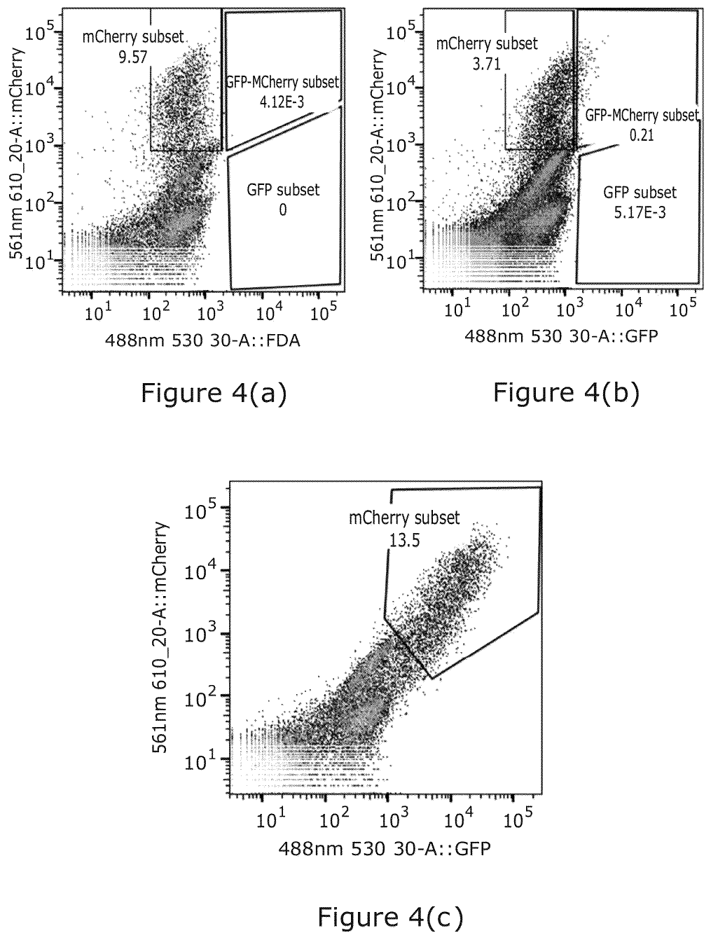

Population observation of transiently transfected A. thaliana protoplast leaf cells showed that there are promoters within the nMYB113 library pool driving expression of GFP that can trigger an increase in green fluorescence in comparison to wild type and there are variants of MYB113 promoter sequence with minimal changes that are able to drive protein expression (see FIG. 4).

A transcriptional activity assay with unique molecular identifiers (UMIs) was used to unravel the individual promoter variants responsible for the increase in gene expression. Specifically for the transcriptional activity assay a library pool of 1250 variations of 754bp 5′ regulatory region of MYB113 gene immediately upstream of 5′ MYB113 UTR was cloned into a proprietary partner sourced SuRE plasmid vector (Arensbergen et al. (2017) Nat Biotechnol. Feb; 35(2): 145-153).

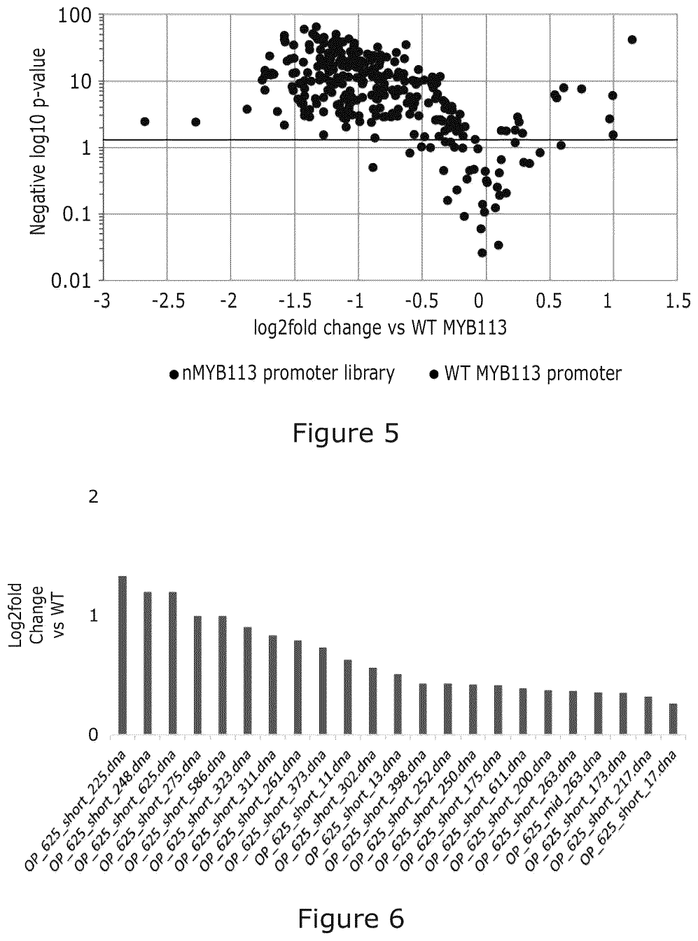

A transcriptional activity assay based on using SuRE plasmids with an nMYB113 promoter element pool has quantified that a portion of the in silico designed library was able to upregulate transcription. Together with UMI barcodes 23 non-wild type promoter variants were identified that have higher transcriptional activity than wild type MYB113 in protoplast cells. This is a key validation before full plant physiological trials. This also demonstrates the advantage of robust high throughput biological validation of AI-designed libraries, as surprisingly only a fraction of the in silico designed library performed as predicted in plant cells (see FIGS. 5 and 6).

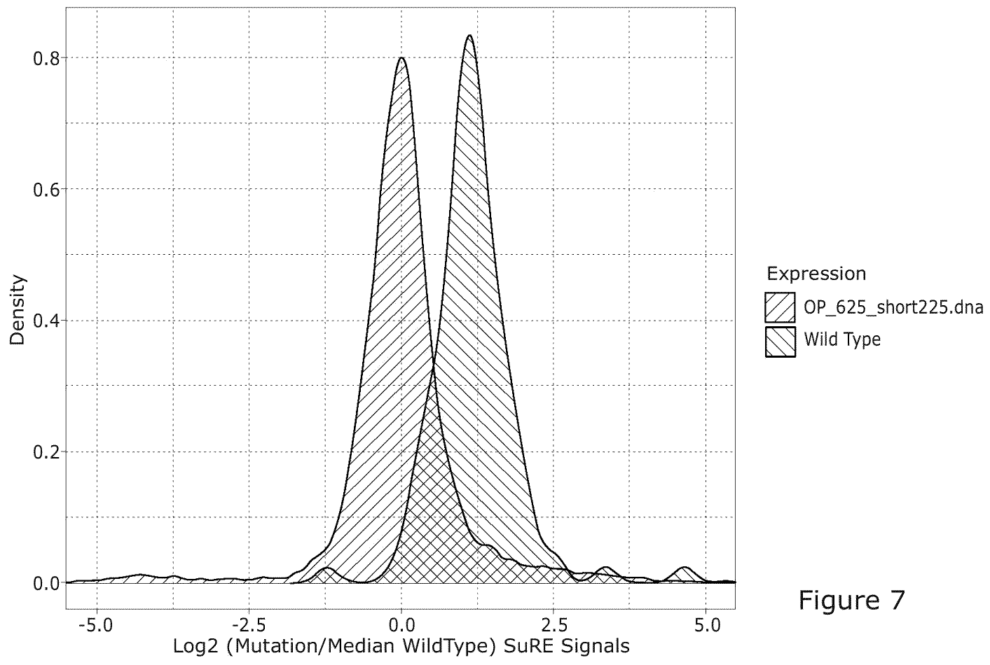

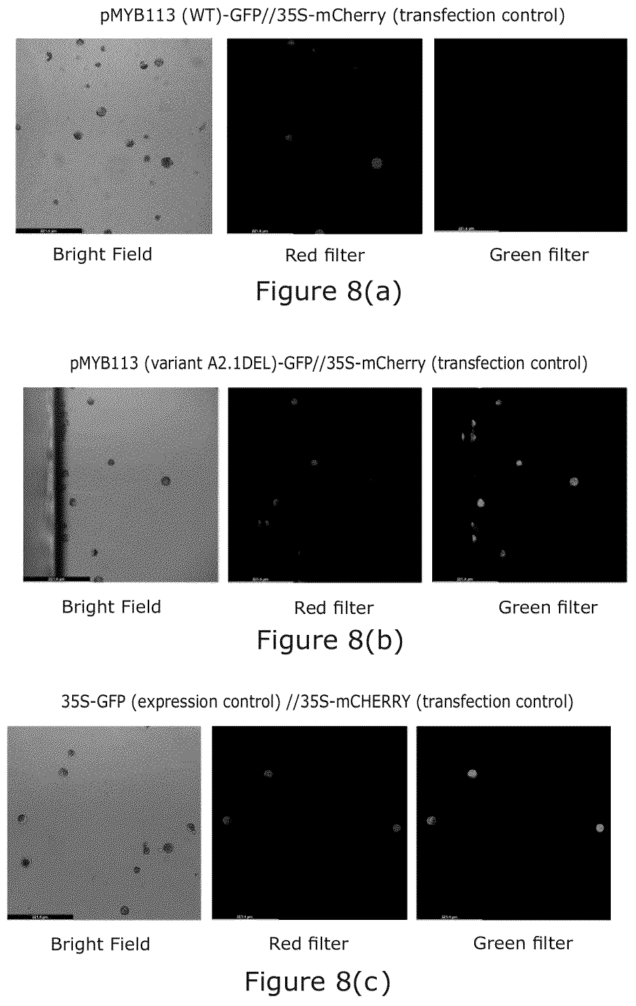

FIG. 7 provides a demonstration of the expression distribution of one of the designed nMYB113 elements referred to as OP625_short_225. The distribution was made using multiple different UMIs. The X-axis indicates log 2fold change in expression with the axis normalised based on the WT MYB113 promoter distribution. The Y-axis shows the density of different UMIs for each element.

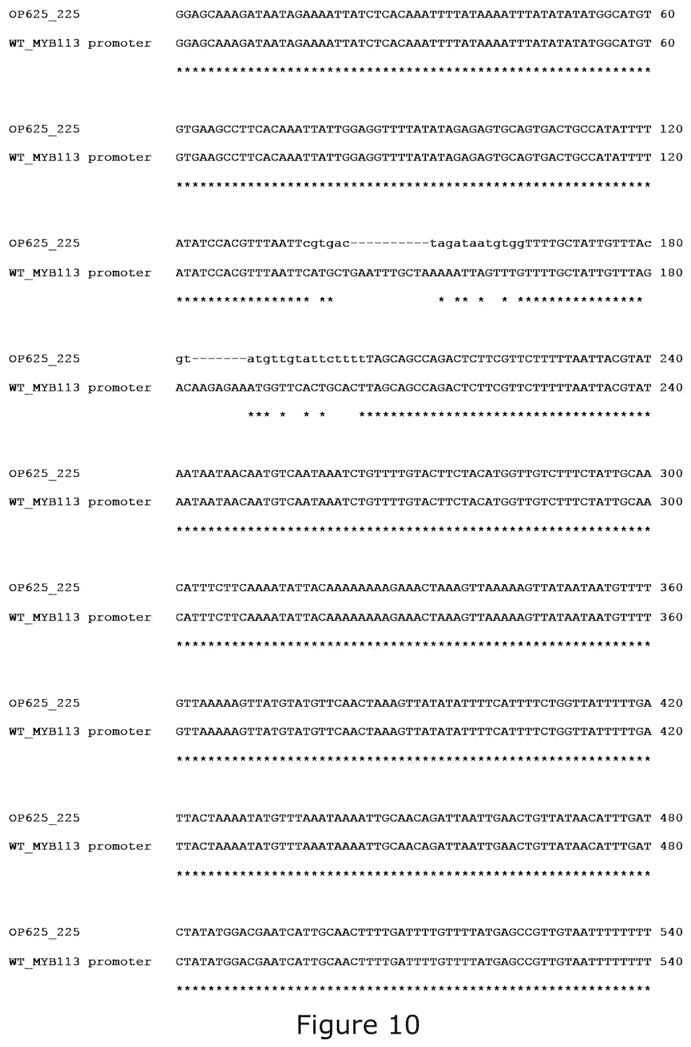

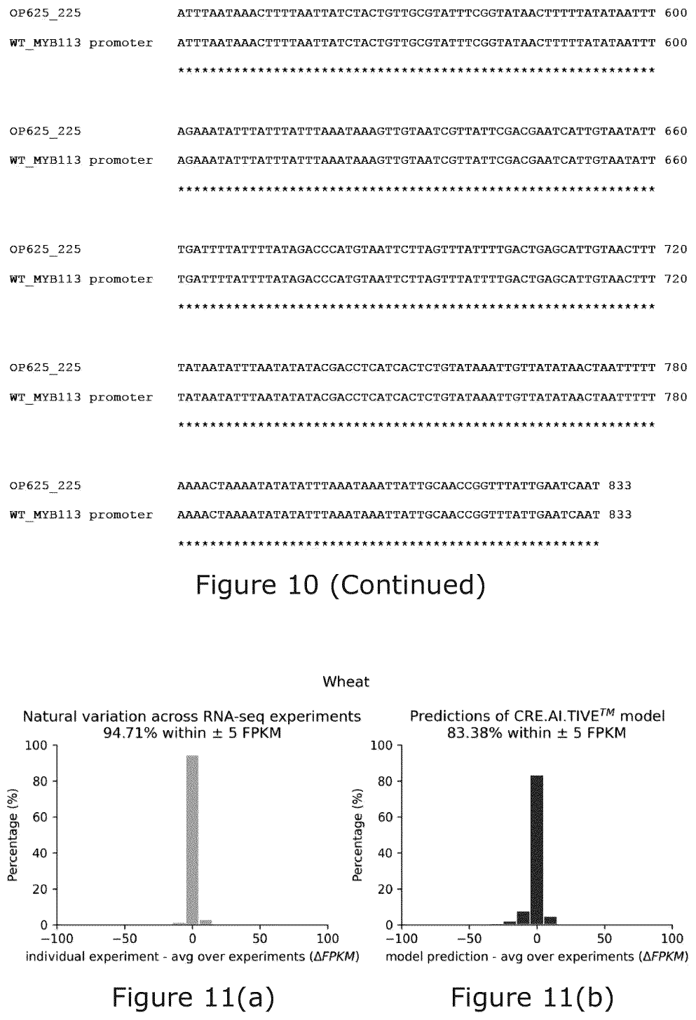

Individual designed promoter elements can be further validated—when taken out of the library pool context and tested at low throughput by fluorescence microscopy observation. One such element in the present experiment, called A2.1DEL, was observed to drive protein expression and create a strong fluorescence signal in transiently transfected protoplast cells in the absence of stress (see FIG. 8(b)). For comparison wild type MYB113 promoter driven GFP expression amounted to negligible fluorescence (FIG. 8(a)). An exemplary variant promoter sequence called OP625_225 (SEQ ID NO:3) is shown aligned to wild type MYB113 (SEQ ID NO: 4) in FIG. 10.

Hence, following high throughput and low throughput confirmation of increased gene expression in Arabidopsis protoplasts, a small subset of the nMYB113 promoter library can be further selected to validate changes in plant physiology in full plants implemented either by stable transformation or genome editing.

### Materials and Methods:

Production of protoplasts. Arabidopsis thaliana protoplasts were obtained using the protocol described in Yoo, Cho, & Sheen, (2007) see above. and washed in buffer twice to remove any free calcium ions. The washed protoplasts were then resuspended in a first solution to a cell concentration of 200 cells/μl. The first solution comprised 100 mM CaCl₂, 100 mM EDTA, 2% w/v Na-alginate and 0.5M Mannitol sterilised by autoclaving.

Transfection: The isolated A. thaliana protoplasts were transfected with plasmids containing either the nMYB113 promoter library or the controls using a PEG-mediated transfection method described by Yoo, Cho and Sheen (2007). The control plasmids have either GFP or an mCHERRY fluorescence gene under a 35s expression cassette. The nMYB113 promoter library was characterised by the presence of a MYB113 promoter variant upstream of a GFP gene and a 35s promoter upstream of an mCHERRY gene, used as an internal control. The transfected protoplasts were incubated overnight in a buffer containing sodium chloride (154 mM), calcium chloride (135 mM), potassium chloride (5 mM), Glucose (5 mM) and MES (1.5 mM) at room temperature, to ensure expression of the fluorescent protein downstream to the promoters.

FACS: Fluorescent activated cell sorting (FACS) was used to sort the transfected cells based on the nature and measure of their fluorescence, using a method previously described by Gronlund et. al. in 2012. The control protoplasts have fluorescence due to either GFP (green) or mCHERRY (red) expression downstream to a 35s promoter. The protoplasts harbouring the nMYB113 library, on the other hand, were sorted based on the GFP expression driven by the activity of these novel promoters as well as mCHERRY expression driven by a 35s promoter, used as an internal control. The sorted cells harbouring active MYB113 promoters were collected for DNA/RNA extraction.

DNA extraction and PCR for Sequencing: DNA was extracted from the sorted protoplasts using the Zymo Quick-DNA Plant/Seed Miniprep Kit (files.zymoresearch.com/protocols/_d6020_quick-dna_plant-seed_miniprep_kit.pdf). This DNA was then used as a template to amplify the MYB113 promoter variants present in the sorted cells, which were sent for next generation sequencing (NGS) for identification.

RNA Extraction, cDNA synthesis and PCR for sequencing: Alternatively, total RNA was extracted from the sorted protoplasts using the Sigma Spectrum plant total RNA extraction kit (www.sigmaaldrich.com/deepweb/assets/sigmaaldrich/product/documents/885/605/strn10bul.pdf). The extracted RNA was used as a template for cDNA synthesis using the NEB ProtoScript® First Strand cDNA Synthesis Kit (international.neb.com/protocols/0001/01/01/first-strand-cdna-synthesis-e6300).

The MYB113 promoter variants present in the sorted cells were amplified from the purified cDNA and sent for NGS for identification.

Transcriptional activity assay: For the transcriptional activity assay, the nMYB113 library was cloned into the SuRE plasmid with unique molecular identifier (UMI) for each of the promoter variants. This library of plasmids was then transfected into the A. thaliana protoplasts using the method described above (Yoo et. al. 2007). Total RNA was then extracted from the transfected protoplasts using the Sigma Spectrum plant total RNA kit and sent to the provider for further transcriptional analysis.

### Example 3—An In Silico Model Based on Convolutional Neural Network and Transformer Architecture can Predict RNA-Seg Count in FPKM or TPM, for Multiple Genes for a Given Plant Tissue and Environmental Condition

A machine learning (ML) model (CRE.AI.TIVE™ v 3.5) has been trained to predict RNA-seq count values for genes in different conditions based upon a dataset comprised of on multiple different genomes of plant species. The accuracy metric considers biological replicate variation. For instance. for a gene A, sampled in a tissue A (e.g. seed), the RNA-seq count will slightly vary between individual seeds. When considering the accuracy of model predictions, it is desirable see how many predictions the model creates that are indistinguishable from natural biological replicate variation. The gene input used to create model predictions was kept as a test set away from the model training, and model weights were not influenced by the test set.

### Wheat

Genomic input: DNA input from annotated reference genome Triticum aestivum (IWSC.v51)—Ensembl Plants
Transcriptome input: RNA-seq datasets (in FPKM) from Plant Public RNA-seq Database (doi.org/10.1111/pbi.13798)
25 RNA-seq datasets
11,311 genes in natural variation dataset
2,273,511 datapoints in natural variation dataset
90,487 genes in training set
11,311 genes in test set
2,544,950 total datapoints (training and validation)

Results: The natural variation in gene expression and the model predicted gene expression is shown in FIG. 11, with 11,311 genes tested for both natural variation and model predictive ability across 25 RNA-seq experiments. FIG. 11(a) shows natural variation of biological replicates with 94.71% genes having their biological replicate RNA-seq count (FPKM) measurements within +/−5FPKM of the mean. FIG. 11(b) shows the model predicted gene expression for genes in the test set, 83.38% genes had a predicted RNA-seq count value within +/−5 FPKM of the mean experimental value.

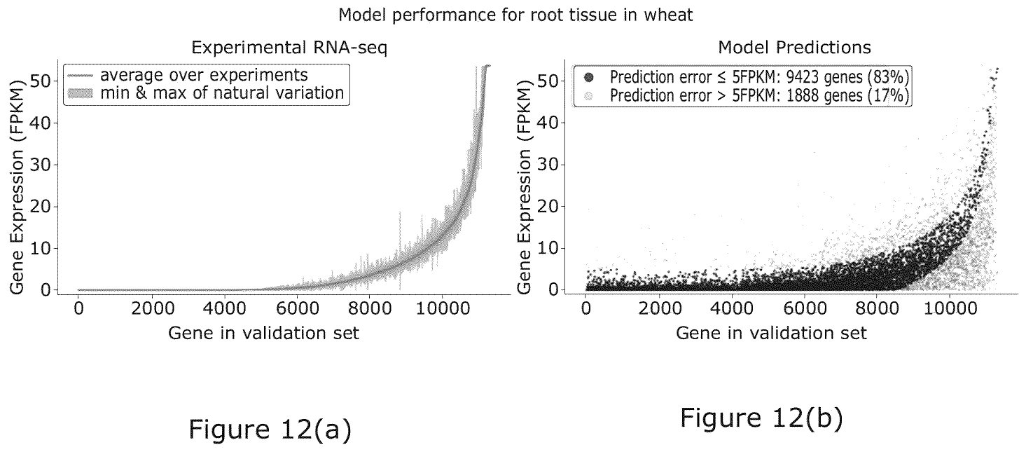

The natural variation in gene expression and the model predicted gene expression is shown in FIG. 12 with 11,311 genes tested for both natural variation and model predictive ability for a single RNA-seq experiment in root tissue. FIG. 12(a) shows the average gene expression for root tissue for 11,311 genes (with three replicates per gene) which is plotted from experimental RNA-seq data. The x-axis shows individual gene expression values for the measured genes with their min/max replicate value, in order of gene expression strength, with lowest expressing to highest expressing from left to right. The y-axis shows the strength of gene expression in FPKM. FIG. 12(b) shows the predictions created by the trained model which are plotted for each individual gene. The dots coloured in dark grey are within 5 FPKM of the average value observed experimentally. The dots coloured in light grey are outside of the 5 FPKM boundary of the average experimentally observed value. Hence, it is apparent that the model predictions align closely to the in vivo expression data.

### Maize

Genomic input: DNA input from annotated reference genome B73-B73.4—Ensembl Plants Transcriptomic input: RNA-seq datasets (in FPKM) from Plant Public RNA-seq Database (doi.org/10.1111/pbi.13798)
116 RNA-seq datasets
4,554 genes in natural variation dataset
1,962,774 datapoints in natural variation dataset
36,435 genes in training set
4,554 genes in test set
4,754,724 total datapoints (training and validation)

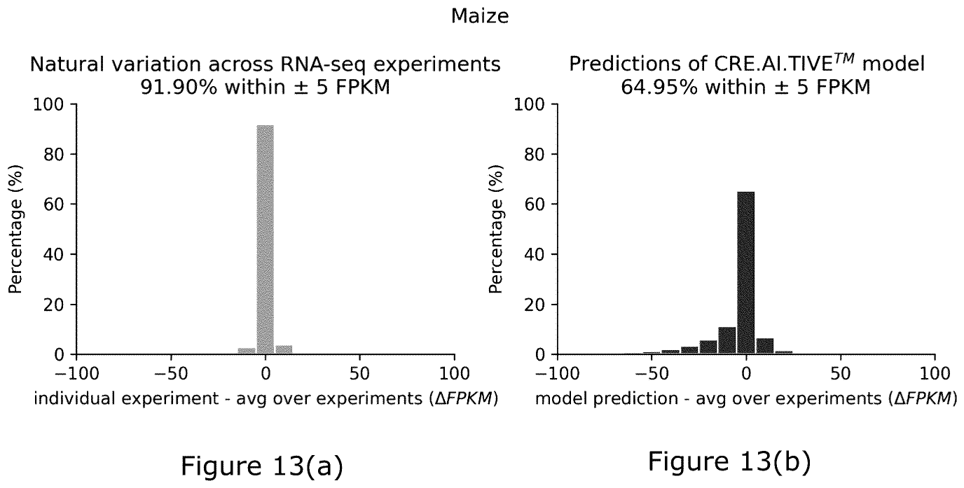

Results: The natural variation in gene expression and the model predicted gene expression is shown in FIG. 13 with 4,554 genes tested for both natural variation and model predictive ability across 116 RNA-seq experiments. FIG. 13(a) shows a natural variation of biological replicates with 91.9% genes had their biological replicate RNA-seq count (FPKM) measurements within +/−5FPKM of the mean. FIG. 13(b) shows the model predicted gene expression for genes in the test set, 64.95% genes had a predicted RNA-seq count value within +/−5 FPKM of the mean experimental value.

### Soybean

Genomic input: DNA input from annotated reference genome Williams 82 (Wm82.a2.v1)—Genome assembly from Soybase, annotation from Ensembl Plants
Transcriptomic input: RNA-seq datasets (in FPKM) from Plant Public RNA-seq Database (doi.org/10.1111/pbi.13798)
152 RNA-seq datasets
5,559 genes in natural variation dataset
2,295,867 datapoints in natural variation dataset
44,835 genes in training set
5,559 genes in test set
7,659,888 total datapoints (training and validation)

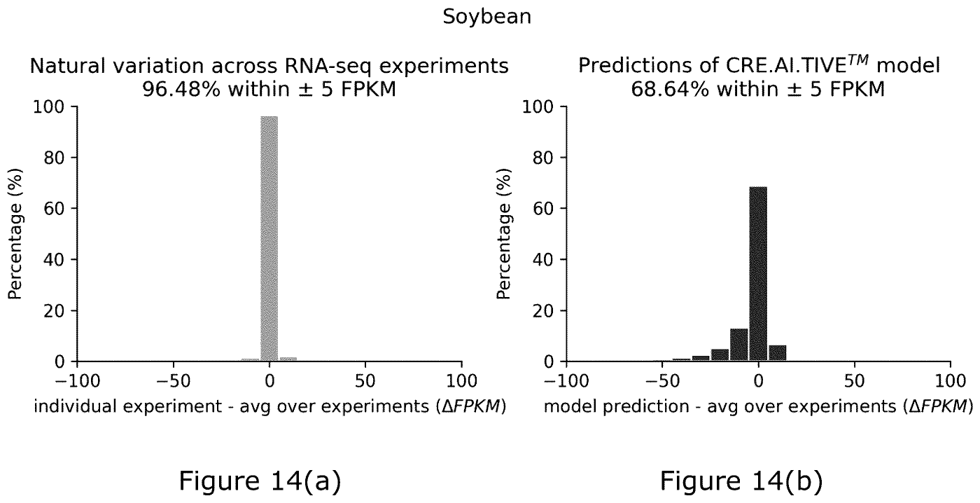

Results: The natural variation in gene expression and the model predicted gene expression is shown in FIG. 14 with 5,559 genes tested for both natural variation and model predictive ability across 152 RNA-seq experiments. In FIG. 14(a) natural variation of biological replicates is shown with 96.48% genes had their biological replicate RNA-seq count (FPKM) measurements within +/−5FPKM of the mean. FIG. 14(b) the model predicted gene expression for genes in the test set, 68.64% genes had a predicted RNA-seq count value within +/−5 FPKM of the mean experimental value.

### CONCLUSION

The results demonstrate a robust gene expression prediction model that can create highly predictive RNA-seq count data purely from gene sequence data. The model has demonstrated this predictive ability across a range of plant species and different tissues and development stages.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

What is claimed is:

1. A method for producing a nucleic acid library that is comprised of a plurality of expression control sequences that are configured to modulate the expression of coding sequence operably linked thereto in a plant cell, the method comprising:
performing an in silico analysis of a genome for a plant subject, wherein the in silico analysis includes identification of a plurality of sequences that collectively define a promoterome of the plant subject, wherein each sequence within the promoterome is comprised of an expression control region that extends from the start codon of an open reading frame to around 100 kilobases 5′ and/or 3′ to the start codon;
obtaining a transcriptome in the form of mRNA expression data for the plant subject;
undertaking an analysis using a sequence-based modelling algorithm in order to provide a prediction value of the level of expression of each protein coding gene comprised within the transcriptome and linking the value to a sequence for a corresponding expression control region comprised within the promoterome;
generating a plurality of non-wild type sequence designs for expression control sequences that are most likely to provide a desired expression profile for an operably linked coding sequence, wherein the plurality of non-wild type sequence designs are informed by the prediction value;
synthesising a plurality of non-wild type expression control sequences that correspond to the plurality of non-wild type sequence designs; and
generating a nucleic acid sequence library that comprises the plurality of non-wild type expression control sequences.

2. The method of claim 1, wherein the expression control region comprises one or more sequence selected from the group consisting of: a promoter; an enhancer; a silencer; a transcription factor binding site; an intron; a transgenic sequence; and a transposon.

3. The method of claim 1, wherein the promoterome comprises all or a part of a protein coding region.

4. The method of claim 1, wherein the promoterome does not comprise a protein coding region.

5. The method of claim 1, wherein each sequence within the promoterome is comprised of an expression control region that extends from the start codon of an open reading frame to less than 100 kilobases 5′ and/or 3′ to the start codon, suitably less than 50 kilobases 5′ and/or 3′ to the start codon.

6. The method of claim 1, wherein the analysis using a sequence-based modelling algorithm comprises training an artificial intelligence (AI) or machine learning (ML) model.

7. The method of claim 6, wherein the AI or ML model comprises a sequence prediction algorithm.

8. The method of claim 7, wherein the sequence prediction algorithm is selected from: an artificial neural network (ANN) algorithm such as those selected from the group consisting of: a convolutional neural network (CNN); and a recurrent neural network (RNN), including a bidirectional RNN, masked language model and a transformer network.

9. The method of claim 1, wherein the desired expression profile prioritises expression control sequences that
provide a higher level of expression than the prediction value; or
provide a lower level of expression than the prediction value; or
wherein the plurality of non-wild type sequence designs are constrained by a minimal change requirement.

10. The method of claim 1, wherein the method further comprises validating one or more of the plurality of non-wild type expression control sequences in vivo.

11. The method of claim 10, wherein the in vivo validation comprises cloning one or more of the plurality of non-wild type expression control sequences into one or more plasmids that comprise a reporter gene whose expression is operably linked to the corresponding non-wild type expression control sequence.

12. The method of claim 11, wherein the in vivo validation comprises undertaking a massively parallel reporter assay (MPRA).

13. The method of claim 12, wherein the MPRA comprises a fluorescence activated cell sorting (FACS) analysis.

14. The method of claim 11, wherein each of the one or more plasmids that comprise a reporter gene further comprises a unique molecular identifier (UMI) sequence.

15. The method of claim 14, wherein the in vivo validation comprises undertaking a transcriptional activity assay that measures the amount of the reporter gene mRNA produced and correlates the amount to the corresponding non-wild type expression control sequence via the UMI.

16. The method of claim 11, wherein the in vivo validation is carried out
within a plant protoplast;
within a plant cell;
within a plant; or
within a part of a plant.

17. The method of claim 1, wherein the plant subject comprises a plant species or plant variety.

18. The method of claim 17, wherein the plant species or plant variety is selected from the group consisting of: Solanum spp. (e.g. S. lycopersicum, S. tuberosum, S. melongena, S. muricatum, S. betaceum); Brassica spp. (e.g. B. oleracea, B. napobrassica, B. napus, B. cretica, B. rupestris and B. rapa); Capsicum spp. (e.g. C. annuum, C. baccatum, C. chinense, C. frutescens, C. pubescens); Lupinus spp. (e.g. L. angustifolius, L. albus, L. mutabilis and L. luteus); Phaseolus spp. (e.g. P. acutifolius, P. coccineus, P. lunatus, P. vulgaris and P. dumosus); Vigna spp (e.g. V. aconitifolia, V. angularis, V. mungo, V. radiata, V. subterranea and V. unguiculata); Vicia faba; Cicer arietinum, Pisum sativum, Lathyrus spp. (e.g. L. sativus and L. tuberosus); Lens spp. (e.g. L. culinaris and L. esculenta); Glycine max; Psophocarpus; Cajanus cajan; Arachis hypogaea; Lactuca spp. (e.g. L. sativa, L. serriola, L. saligna, L. virosa and L. taterica); Asparagus officinalis; Apium graveolens; Allium spp. (e.g. A. cepa, A. oschaninii, A. ampeloprasum, A. wakegi, A. porrum, A. sativum and A. schoenoprasum); Beta vulgaris; Cichorium intybus; Taraxacum officinale, Eruca spp. (e.g. E. vesicaria and E. sativa); Cucurbita spp. (e.g. C. argyosperma, C. digitata, C. pepo, C. moschata, C. ecuadorensis, C. ficifolia, C. foetidissima, C. galeottii, C. lundelliana, C. maxima, C. moshata, C. pedatifolia, C. radicans); Spinacia oleracea; Nasturtium officinale; Cucumis spp. (e.g. C. sativus, C. melo, C. hystrix, C. picrocarpus and C. anguria); Olea europaea; Daucus carota; Ipomoea batatas; Ipomoea eriocarpa; Manihot esculenta; Zingiber officinale; Armoracia rusticana; Helianthus spp. (e.g. H. annuus and H. tuberosus); Cannabis spp. (e.g. C. sativa and C. indica); Pastinaca sativa; Raphanus sativus; Curcuma longa; Dioscorea spp. (e.g. D. rotundata, D. alata, D. polystachya, D. bulbifera, D. esculenta, D. dumetorum, D. trifida and D. cayennensis); Piper spp. (e.g. P. aduncum, P. guineense and P. nigrum); Zea spp. (e.g. Z. mays and Z. diploperennis); Hordeum spp. (e.g. H. vulgare, H. pusillum, H. murinum, H. marinum, H. jubatum and H. intercedens); Gossypium spp. (e.g. G. hirsutum, G. barbadense, G. arboreum and G. herbaceum); Triticum spp. (e.g. T. aestivum and T. timopheevii); Vitis vinifera; Prunus sp. (e.g. P. avium, P. armeniaca, P. cerasifera, P. cerasus, P. domestica, P. persica and P. dulcis); Malus domestica; Pyrus spp. (e.g. P. communis, P. cordata and P. pyrifolia); Fragaria vesca and Fragaria x ananassa; Rubus idaeus; Saccharum officinarum; Sorghum saccharatum; Musa balbisiana and Musa x paradisiaca; Oryza sativa; Nicotiana tabacum; Arabidopsis thaliana; Citrus spp. (e.g. C. x aurantiifolia, C. x aurantium, C. x latifolia, C. x limon, C. x limonia, C. x paradisi, C. x sinensis and C. x tangerina); Populus spp. (e.g. P. tremula, P. balsamifera and P. tomentosa); Tulipa gesneriana; Medicago sativa; Abies balsamea; Avena orientalis; Bromus mango; Calendula officinalis; Chrysanthemum balsamita; Dianthus caryophyllus; Eucalyptus spp. (e.g. E. leucoxylon, E. maculata, E. polybractea, E. sargentii); Impatiens biflora; Linum usitatissimum; Lycopersicon esculentum; Mangifera indica; Nelumbo spp. (e.g. N. nucifera and N. pentapatala); Poaceae spp.; Secale cereale; Tagetes erecta; and Tagetes minuta.

19. A nucleic acid library that comprises a plurality of non-wild type expression control sequences that are configured to modulate the expression of coding sequence operably linked thereto within a plant cell, wherein the plurality of non-wild type expression control sequences is generated by the method as defined within claim 1.


SEQUENCE LISTING (4 sequences)

SEQ ID NO: 1 - DNA, 5033 bp, synthetic construct
  source 1..5033: mol_type = other DNA
caaccttcagattgtgttttccgattaactaatcgttggatcgggctgaaatttgtactgagtgttcatgacatcttggtgattgatttaaatggtggtgatcggattcggaggttagcaagattctgttttaggtcccgaacagtagctgggtttgggtggtgtttctttctatttatcttttgttggtgtagctattgtttgttctcttttggcacttattgtgtagccattagaggaataattgtaactctcttattgttatagtgaagcaattcggatcttgtcgatcccgtggttgttaccttcgttttgaaggatttttccacgttaaacttggtgttctttattttagattttcgttttcatatttttcgtcataacacaaattggatatgttttatttctgtatatttaaaccaaaatagaagagttagtcttttgctacttaacagaaatatttccttgttgataacatattaacagttatgttatgggagctaccaaacaaaaattaaacaaacttcaaatcttatgcacttaagagtatatattcttgactcacttggatctttactattcttgatgccaatgaaaataatgttagtcttttgttacttcatcaaaatcttttcatgtttatcaaaagagaataatgatgatagcatattaatagctattatttgggagctaccaaagaaaaatccaataaaaattctctaacaaattcaaaatgtttcagcagatagagggagcaaaacctttctagtatattagagaaacccaaaatgcaccagtttggagcatgaactaacctgcgccagtttcagcctctaaggagggagtatactatcttctagatttcttgatgattgtgtctctcgagatgttctttggcgatagctacttgatatttgttctgtttgttgaggtacttgacatggattagatgattgtcttgtctctaaatatacctttgtaactcctattggttgtacttgtggttgcgactttatttcagaacttccacgaccttgaaaattgaaacatccagatcatgtaccacccctcccacttttaccttgtccagttcctttcattttcaccaacatcagattcaaaaaagagaagtataaaaatcagaaattaatcaaagacaaactaatgtgaaaagtaagtctatggtaaataacacaataatctctggacccattgacaaacaaagacaaactcatcataaaataaaacgagtttgaccaactaatgaaattaagtatatgatgtggctttatgttatttatttcctgtacatatttcaattcaaatagactatattgttgtattggaataaattaattaaccacaacacatgtaagcatggcgtaacttcaaaccaaaaacaaaaggtttcagctgggaaagtgaatatatacaagttcactccccccagtgagcgaggtattcccactcgtgaagatggataaagtcatttcattttccatcaataggccggcttcatcgcggactcaacaagagaaaaatgaacaagaggagatgttaacgttcgataacataaaatatgataaatagagggtatataaggttcgatgtgtttctgaacgtggacaataatgtgaacgcgcatgagcttgataaggcagagttcgcggggagttatactagtttgccacatgttcacggaattggcaagaatgatcaaagcccgcctgttactttccagctggcgataacagaactgttggaggacattggtttggaagatgaagagactatttcggtgactctggtaccaaagaaaggtggtgaacgtatatatattgaaaatgtggagatcaagcttgaggattgttaaagtttcaattgaatctgttgagattacaactttcatatgtttatgtttccttgttctgttttttacttttgtttttccatgtaacttttcctgttgaaatcagcttcatgcttgatttccttggagttgttattaattcactaataaaatcagttactattatgtggtatgtgttcacgatactcccgacatttacataaaatcaacgatcgatgatgtaattgacgtaaaaataatgatgtttgccgtaaaatatcgttaaaataatattattttatacaaatgttaaaaatattgatatttatttcaaatgacaaaaatattatgacatgtattttaaatgtgataatattagtagttaattacagacatttacatttaatattaaataaaaatttaggtcttttcaaattatttttgtcactctagaagaaaaattattggatgcatacataataggagtcttcgtttatttatttttagtttattgatggagtaatctttatagtaataaatcagaaattataaaattaagggaatcgtagctcgaccctttgttccaattagcaaatgaggactgcttatatatataaaaaagaggcaatcaaaaaaaaacatgtagaaattactcaatctttgtacaaatattcaagtcgagggcttaaatagacaaaaataatagaatcataagtggaccattatttttatataaaccgattttttttctttatttatgttgaggagacactatttgtatgagacctaatctataacattagatgactatggactaaagatttttacatgcaaaaatttcaaatgagagcttagtaattatttagatatgattatctacctatattacacttaatcggatatgtacttctctataattatattgatcaccaattatatttaatttaaataaacatcaaaattataattttacgatctattaacttatcatgacttatttttttaagttaaaataaaaaataaatcttaaacttctaacttatactaataaactataagtttatccaaaaatgctcccagactattttaataggacgaagtggacaaaagagtagtaggaatgaagaaagaaattggatcttgacactatagtctctctctatatatatggtgatcctatttgcatttttgcgtgtttacttatattttaataaaaaaaattgactctactatttaaattcacaaatggtcactcaattatgaacctttcagtcaataccaataataatttttttactcaaaaattacttaactatagattttttctctcaaaatcactcaattttgatttttaactcaaaaatcactcaactatgattattttatttacgaagtcactcaaactatttaattgatttttgaaatttaaatttattgacatgaaatttttatatctaaccaaaattttaaaaaataatatatccatttagtgacccacccactcagaccgacccgctaaaaatgtaatattaacctttttttatatttaaaagaatactttcttatttcttaaaagttggattagaaataaaaaatcatatcaacagattttaattaaaaaaatcaattaaataaattcaatgactttataagtaaaacagtcatataattaaatgacttttaaattataaatcaaaattagagtaaattttagaaataaaaatttacaattaaataagtatatgagatattatctcaacggttcaattatctttctatgaacattcatttccgacaactggtactaaaagctctacatctcctagtgggaaaccacatggacctcacgacaaaaacacttttttcaaaagaatattgtttgcaattatatgccaaaagatctacattaatagaacatgccattatggcgtagctagaagtctgattacagatttagttaaattcaacaatatttacttaaataatatatttatatagtaattttttttataaaatatatacaaattaagctaaagaattcaattactaaccccttaaaattgtattataaattcaatgttaaaattttaatttctcccgtgcttaacattacttatcaaatttttgtttctatgtagaaaagtactactactcttttgactcgtctagatgtctcctatagatctcctaataagactaaaatttccagttttaagattttcctcacaattattattcttaatctcccatgcacctaccgagtcacacatatattaaatgagcctctaataaatttgctccaattggggtatattatatctattaatttatgatctacctaatgataatttgtaatctagttaaaatgatggcaaaaaaaatataatatctagactgaagttcttagtcaatagcgtaaatgaaagaaaaaaaaagctcaagaagaaacatgatatcttgttgctctgattcgtaaaaaaaacataataacttcataaaatatcttatcttttgaacagagcgtataacaataataatgaaaaaaataaaatatattactagtaatactaagattagttacctgagactgtttcctatcttttgtttgatttgatttattaaggacaattatgtttcaacggccatgcttatccatgcattattgatgatcaatatattattaaatgctattactatagtttgccacgtacttcctctatctctaattatttgtccgcttttgaattgacgaatttaccattttatctctattaattatgaagtggatgaaaataagattaattattctacatttttcaaaataattagtcatttaattgagagaataataggtaaaaagaatttgtcctttcttgatttgtcaaaatggacaagtaattagggacaaagggagtattaattatatgttcttctgtcaaccgacccaaccgacccctaaagagattaattaaggttgattgccatgatgagcccagactgaagttatttttctgtttactacagagtcttccaagaacaaaaacttaataattgtatggctgctatacaaaattccccacctaccacttcctggaataattgatatggaagccgcctctaaaattgaataattatattgttttacatattatataaggcaaggtatagccaaatgaattttcat

SEQ ID NO: 2 - DNA, 4992 bp, Solanum tuberosum
  source 1..4992: mol_type = genomic DNA
caaccttcagattgtgttttccgattaactaatcgttggatcgggctgaaatttgtactgagtgttcatgacatcttggtgattgatttaaatggtggtgatcggattcggaggttagcaagattctgttttaggtcccgaacagtagctgggtttgggtggtgtttctttctatttatcttttgttggtgtagctattgtttgttctcttttggcacttattgtgtagccattagaggaataattgtaactctcttattgttatagtgaagcaattcggatcttgtcgatcccgtggttgttaccttcgttttgaaggatttttccacgttaaacttggtgttctttattttagattttcgttttcatatttttcgtcataacacaaattggatatgttttatttctgtatatttaaaccaaaatagaagagttagtcttttgctacttaacagaaatatttccttgttgataacatattaacagttatgttatgggagctaccaaacaaaaattaaacaaacttcaaatcttatgcacttaagagtatatattcttgactcacttggatctttactattcttgatgccaatgaaaataatgttagtcttttgttacttcatcaaaatcttttcatgtttatcaaaagagaataatgatgatagcatattaatagctattatttgggagctaccaaagaaaaatccaataaaaattctctaacaaattcaaaatgtttcagcagatagagggagcaaaacctttctagtatattagagaaacccaaaatgcaccagtttggagcatgaactaacctgcgccagtttcagcctctaaggagggagtatactatcttctagatttcttgatgattgtgtctctcgagatgttctttggcgatagctacttgatatttgttctgtttgttgaggtacttgacatggattagatgattgtcttgtctctaaatatacctttgtaactcctattggttgtacttgtggttgcgactttatttcagaacttccacgaccttgaaaattgaaacatccagatcatgtaccacccctcccacttttaccttgtccagttcctttcattttcaccaacatcagattcaaaaaagagaagtataaaaatcagaaattaatcaaagacaaactaatgtgaaaagtaagtctatggtaaataacacaataatctctggacccattgacaaacaaagacaaactcatcataaaataaaacgagtttgaccaactaatgaaattaagtatatgatgtggctttatgttatttatttcctgtacatatttcaattcaaatagactatattgttgtattggaataaattaattaaccacaacacatgtaagcatggcgtaacttcaaaccaaaaacaaaaggtttcagctgggaaagtgaatatatacaagttcactccccccagtgagcgaggtattcccactcgtgaagatggataaagtcatttcattttccatcaataggccggcttcatcgcggactcaacaagagaaaaatgaacaagaggagatgttaacgttcgataacataaaatatgataaatagagggtatataaggttcgatgtgtttctgaacgtggacaataatgtgaacgcgcatgagcttgataaggcagagttcgcggggagttatactagtttgccacatgttcacggaattggcaagaatgatcaaagcccgcctgttactttccagctggcgataacagaactgttggaggacattggtttggaagatgaagagactatttcggtgactctggtaccaaagaaaggtggtgaacgtatatatattgaaaatgtggagatcaagcttgaggattgttaaagtttcaattgaatctgttgagattacaactttcatatgtttatgtttccttgttctgttttttacttttgtttttccatgtaacttttcctgttgaaatcagcttcatgcttgatttccttggagttgttattaattcactaataaaatcagttactattatgtggtatgtgttcacgatactcccgacatttacataaaatcaacgatcgatgatgtaattgacgtaaaaataatgatgtttgccgtaaaatatcgttaaaataatattattttatacaaatgttaaaaatattgatatttatttcaaatgacaaaaatattatgacatgtattttaaatgtgataatattagtagttaattacagacatttacatttaatattaaataaaaatttaggtcttttcaaattatttttgtcactctagaagaaaaattattggatgcatacataataggagtcttcgtttatttatttttagtttattgatggagtaatctttatagtaataaatcagaaattataaaattaagggaatcgtagctcgaccctttgttccaattagcaaatgaggactgcttatatatataaaataaaaaaaaacatgtagaaattactcaatctttgtacaaatattcaagtcgagggcttaaatagacaaaaataatagaatcataagtggaccattatttttatataaaccgattttttttctttatttatgttgaggagacactatttgtatgagacctaatctataacattagatgactatggactaaagatttttacatgcaaaaatttcaaatgagagcttagtaattatttagatatgattatctacctatattacacttaatcggatatgtacttctctataattatattgatcaccaattatatttaatttaaataaacatcaaaattataattttacgatctattaacttatcatgacttatttttttaagttaaaataaaaaataaatcttaaacttctaacttatactaataaactataagtttatccaaaaatgctcccagactattttaataggacgaagtggacaaaagagtagtaggaatgaagaaagaaattggatcttgacactatagtctctctctatatatatggtgatcctatttgcatttttgcgtgtttacttatattttaataaaaaaaattgactctactatttaaattcacaaatggtcactcaattatgaacctttcagtcaataccaataataatttttttactcaaaaattacttaactatagattttttctctcaaaatcactcaattttgatttttaactcaaaaatcactcaactatgattattttatttacgaagtcactcaaactatttaattgatttttgaaatttaaatttattgacatgaaatttttatatctaaccaaaattttaaaaaataatatatccatttagtgacccacccactcagaccgacccgctaaaaatgtaatattaacctttttttatatttaaaagaatactttcttatttcttaaaagttggattagaaataaaaaatcatatcaacagattttaattaaaaaaatcaattaaataaattcaatgactttataagtaaaacagtcatataattaaatgacttttaaattataaatcaaaattagagtaaattttagaaataaaaatttacaattaaataagtatatgagatattatctcaacggttcaattatctttctatgaacattcatttccgacaactggtactaaaagctctacatctcctagtgggaaaccacatggacctcacgacaaaaacacttttttcaaaagaatattgtttgcaattatatgccaaaagatctacattaatagaacatgccattatggcgtagctagaagtctgattacagatttagttaaattcaacaatatttacttaaataatatatttatatagtaattttttttataaaatatatacaaattaagctaaagaattcaattactaaccccttaaaattgtattataaattcaatgttaaaattttaatttctcccgtgcttaacattacttatcaaatttttgtttctatgtagaaaagtactactactcttttgactcgtctagatgtctcctatagatctcctaataagactaaaatttccagttttaagattttcctcacaattattattcttaatctcccatgcacctaccgagtcacacatatattaaatgagcctctaataaatttgctccaattggggtatattatatctattaatttatgatctacctaatgataatttgtaatctagttaaaatgatggcaaaaaaaatataatatctagactgaagttcttagtcaatagcgtaaatgaaagaaaaaaaaagctcaagaagaaacatgatatcttgttgctctgattcgtaaaaaaaacataataacttcataaaatatcttatcttttgaacagagcgtataacaataataatgaaaaaaataaaatatattactagtaatactaagattagttacctgagactgtttcctatcttttgtttgatttgatttattaaggacaattatgtttcaacggccatgcttatccatgcattattgatgatcaatatattattaaatgctattactatagtttgccacgtacttcctctatctctaattatttgtccgcttttgaattgacgaatttaccattttatctctattaattatgaagtggatgaaaatttctacatttttcaaaataattagtcatttaattgagagaataataggtaaaaagaatttgtcctttcttgatttgtcaaaatggacaagtaattagggacaaagggagtattaattatatgttcttctgtcaaccgacccaaccgacccctaaagagattaattatccagactgaagttatttttctgtttactacagagtcttccaagaacaaaaacttaataattgtatggctgctatacaaaattccccacctaccacttcctggaataattgatatggaagccgcctctaaaattgaataattatattgttttacatattatataaggcaaggtatagccaaatgaattttcat

SEQ ID NO: 3 - DNA, 816 bp, synthetic construct
  source 1..816: mol_type = other DNA
ggagcaaagataatagaaaattatctcacaaattttataaaatttatatatatggcatgtgtgaagccttcacaaattattggaggttttatatagagagtgcagtgactgccatattttatatccacgtttaattcgtgactagataatgtggttttgctattgtttacgtatgttgtattctttttagcagccagactcttcgttctttttaattacgtataataataacaatgtcaataaatctgttttgtacttctacatggttgtctttctattgcaacatttcttcaaaatattacaaaaaaaagaaactaaagttaaaaagttataataatgttttgttaaaaagttatgtatgttcaactaaagttatatattttcattttctggttatttttgattactaaaatatgtttaaataaaattgcaacagattaattgaactgttataacatttgatctatatggacgaatcattgcaacttttgattttgttttatgagccgttgtaattttttttatttaataaacttttaattatctactgttgcgtatttcggtataactttttatataatttagaaatatttatttatttaaataaagttgtaatcgttattcgacgaatcattgtaatatttgattttattttatagacccatgtaattcttagtttattttgactgagcattgtaacttttataatatttaatatatacgacctcatcactctgtataaattgttatataactaatttttaaaactaaaatatatatttaaataaattattgcaaccggtttattgaatcaat

SEQ ID NO: 4 - DNA, 833 bp, Arabidopsis thaliana
  source 1..833: mol_type = genomic DNA
ggagcaaagataatagaaaattatctcacaaattttataaaatttatatatatggcatgtgtgaagccttcacaaattattggaggttttatatagagagtgcagtgactgccatattttatatccacgtttaattcatgctgaatttgctaaaaattagtttgttttgctattgtttagacaagagaaatggttcactgcacttagcagccagactcttcgttctttttaattacgtataataataacaatgtcaataaatctgttttgtacttctacatggttgtctttctattgcaacatttcttcaaaatattacaaaaaaaagaaactaaagttaaaaagttataataatgttttgttaaaaagttatgtatgttcaactaaagttatatattttcattttctggttatttttgattactaaaatatgtttaaataaaattgcaacagattaattgaactgttataacatttgatctatatggacgaatcattgcaacttttgattttgttttatgagccgttgtaattttttttatttaataaacttttaattatctactgttgcgtatttcggtataactttttatataatttagaaatatttatttatttaaataaagttgtaatcgttattcgacgaatcattgtaatatttgattttattttatagacccatgtaattcttagtttattttgactgagcattgtaacttttataatatttaatatatacgacctcatcactctgtataaattgttatataactaatttttaaaactaaaatatatatttaaataaattattgcaaccggtttattgaatcaat